(19) United States
(12) Patent Application Publication
(10) Pub. No.: US 2007/0088082 A1
(43) Pub. Date: Apr. 19, 2007

(54) Polymorphic forms of ladostigil tartrate

(76) Inventors: Judith Aronhime, Rehovot (IL); Eliezer Bahar, Tel-Aviv (IL); Anton Frenkel, Modi'in (IL); Ronen Gottesfeld, Netanya (IL); Amir Gold, Herzelia (IL); Tamas Koltai, Netanya (IL)

Correspondence Address: KENYON & KENYON LLP, ONE BROADWAY, NEW YORK, NY, 10004, US

(21) Appl. No.: 11/541,152

(22) Filed: Sep. 28, 2006

Related U.S. Application Data
(60) Provisional application No. 60/721,714, filed on Sep. 28, 2005.

(51) Int. Cl. A61K 31/325; C07C 261/02

(52) U.S. Cl. 514/490; 560/134

(57) ABSTRACT

Provided are polymorphic forms of ladostigil tartrate and methods for preparation thereof.

### RELATED APPLICATIONS

This application claims the benefit of U.S. Provisional Application No. 60/721,714, filed Sep. 28, 2005.

### FIELD OF THE INVENTION

The invention relates to solid state chemistry of ladostigil tartrate, particularly polymorphic forms of ladostigil tartrate and processes for the preparation thereof.

### BACKGROUND OF THE INVENTION

Ladostigil is an active pharmaceutical ingredient which has shown to be effective in animal models of Alzheimer's disease. It contains a (R)—N-propargyl aminoindan moiety which is a monoamine oxidase type B inhibitor. It also contains a carbamate moiety which is effective as an acetylcholine esterase inhibitor. Ladostigil is disclosed in Weinstock, M. et al: J Neuronal Transm. (2000) [suppl]; 60: 157-169, Weinstock, M. et al: Development Research (2000); 50:216-222, Sterling J. et al: J. Med. Chem. 2002; 45:5260-5279, Weinstock M. et al: Psychopharmacology 2002;160:318-324; and Yogev-Falach et al: FASEB J. 2002; October 16(12):1674-1676.

The chemical name of ladostigil tartrate is carbamic acid, ethylmethyl-, (3R)-2,3-dihydro-3-(2-propynylamino)-1H-inden-5-yl ester, (2R,3R)-2,3-dihydroxybutanedioate (2:1). Its chemical structure is:

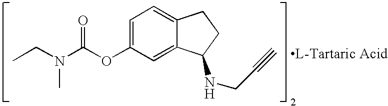

Ladostigil tartrate and a method for its preparation are disclosed in U.S. Pat. No. 6,303,650, hereby incorporated by reference. The '650 patent discloses the preparation of ladostigil tartrate by crystallization in isopropanol.

The occurrence of different crystal forms (polymorphism) is a property of some molecules and molecular complexes. A single molecule, or a salt complex, may give rise to a variety of solids having distinct physical properties like melting point, X-ray diffraction pattern, infrared absorption fingerprint and NMR spectrum. The crystalline form may give rise to thermal behavior different from that of the amorphous material or another crystalline form. Thermal behavior is measured in the laboratory by such techniques as capillary melting point, thermogravimetric analysis (“TGA”) and differential scanning calorimetry (“DSC”) and can be used to distinguish some polymorphic forms from others. The differences in the physical properties of different crystalline forms result from the orientation and intermolecular interactions of adjacent molecules (complexes) in the bulk solid. Accordingly, polymorphs are distinct solids sharing the same molecular formula yet having distinct advantageous and/or disadvantageous physical properties compared to other forms in the polymorph family. These properties can be influenced by controlling the conditions under which the salt is obtained in solid form.

Exemplary solid state physical properties include the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment.

The discovery of new forms of a pharmaceutically useful compound provides an opportunity to improve the performance characteristics of a pharmaceutical product. It enlarges the repertoire of materials that a formulation scientist has available for designing, for example, a pharmaceutical dosage form of a drug with a targeted release profile or other desired characteristic. Additional polymorphic forms may further help in determination of polymorphic content of a batch of an active pharmaceutical ingredient, for example, by providing a useful reference standard for XRD instruments.

The ladostigil tartrate salt obtained in the '650 patent is reported to have a melting point of 143-45° C., but the solid state properties of the salt are not otherwise disclosed. Repetition of the procedure carried out in the '650 patent resulted in ladostigil tartrate Form A1. Crystalline form A1 is characterized in FIGS. 1-4. The differential scanning calorimetric (DSC) thermogram of form A1 is characterized by an endothermic peak at about 147° C. followed by a wide exothermic peak. The exothermic peak most probably represents decomposition of the compound. The water content of the sample is about 0.3% water by weight. The loss upon drying as determined by TGA is 0.3% by weight. Form A1 is anhydrous.

### SUMMARY OF THE INVENTION

One embodiment of the invention provides crystalline ladostigil tartrate (Form A) characterized by an x-ray diffraction pattern having peaks at 19.5, 22.9, and 23.1±0.2 degrees two theta.

Another embodiment of the invention provides a process for preparing crystalline ladostigil tartrate form A including crystallizing ladostigil tartrate from a solution of ladostigil tartrate in water, tetrahydrofuran, isopropanol, methylene chloride, or mixtures thereof, and recovering the crystalline form.

Another embodiment of the invention provides a process for preparing crystalline ladostigil tartrate form A including crystallizing ladostigil tartrate from a solution of ladostigil tartrate in ethanol, acetonitrile, dioxane, or mixtures thereof, to obtain a wet crystal, heating the wet crystal to obtain the crystalline form, and recovering the crystalline form.

Another embodiment of the invention provides a crystalline form of ladostigil tartrate, wherein the crystalline form does not transform to another crystalline form after exposure to air having, relative humidity of 100% for 10 days.

Another embodiment of the invention provides crystalline ladostigil tartrate (Form B), characterized by an x-ray diffraction pattern having peaks at 4.3, 5.6, 11.2, 13.0, 16.8, and 19.9±0.2 degrees two theta.

Another embodiment of the invention provides a process for preparing crystalline ladostigil tartrate form B including crystallizing ladostigil tartrate from a solution of ladostigil tartrate in a C₁ to C₄ alcohol by rapid precipitation and recovering the crystalline form.

Another embodiment of the invention provides a process for preparing crystalline ladostigil tartrate form B including crystallizing ladostigil tartrate from a solution of ladostigil tartrate in 2-propanol by combining the solution with a solid surface having a temperature below that of the solution to precipitate the crystalline form within about 1 hour of combining, and recovering the crystalline form.

Another embodiment of the invention provides a process for preparing crystalline ladostigil tartrate form B including crystallizing ladostigil tartrate from a solution of ladostigil tartrate in a C₁ to C₄ alcohol by combining the solution with an anti-solvent to precipitate the crystalline form within about 1 hour of combining, and recovering the crystalline form.

Another embodiment of the invention provides crystalline ladostigil tartrate (Form C), characterized by an x-ray diffraction pattern having peaks at 5.8, 10.8, 13.3, 17.4, 23.6±0.2 degrees two theta.

Another embodiment of the invention provides a process for preparing crystalline ladostigil tartrate Form C including crystallizing ladostigil tartrate from a solution of ethanol and recovering the crystalline form.

Another embodiment of the invention provides crystalline ladostigil tartrate (Form E), characterized by an x-ray diffraction pattern having peaks at 4.9, 8.7, 12.0, 13.6, and 18.9±0.2 degrees two theta.

Another embodiment of the invention provides a process for preparing crystalline ladostigil tartrate Form E including lyophilizing the crystalline form from an aqueous solution of ladostigil tartrate.

Another embodiment of the invention provides crystalline ladostigil tartrate (Form F), characterized by an x-ray diffraction pattern having peaks at 3.3, 6.4, 13.0, 13.3, and 19.6±0.2 degrees two theta.

Another embodiment of the invention provides a process for crystalline ladostigil tartrate Form F including slurrying ladostigil tartrate form A1 in DMF, acetone, hexane, acetonitrile, or mixtures thereof to obtain the crystalline form, and recovering the crystalline form.

Another embodiment of the invention provides a process for crystalline ladostigil tartrate Form F including crystallizing the crystalline form of ladostigil tartrate from a solution of ladostigil tartrate in DMF, acetone, acetonitrile, or mixtures thereof, by combining the solution with a C₅ to C₇ saturated hydrocarbon and recovering the crystalline form.

Another embodiment of the invention provides crystalline ladostigil tartrate (Form H), characterized by an x-ray diffraction pattern with peaks at 4.4, 8.5, 10.5, 15.6 and 17.7-±0.2 degrees two theta.

Another embodiment of the invention provides a process for preparing crystalline ladostigil tartrate Form H including crystallizing the crystalline form of ladostigil tartrate from a solution of ladostigil tartrate in dioxane and recovering the crystalline form.

Another embodiment of the invention provides crystalline ladostigil tartrate (Form I), characterized by an x-ray diffraction pattern with peaks at 6.5, 12.0, 13.0, 13.3 and 18.6±0.2 degrees two theta.

Another embodiment of the invention provides a process for preparing crystalline ladostigil tartrate Form I including maintaining a heterogeneous mixture of ladostigil tartrate in acetonitrile to obtain the crystalline form and recovering the crystalline form.

Another embodiment of the invention provides crystalline ladostigil tartrate (Form J), characterized by an x-ray diffraction pattern having peaks at 6.3, 12.2, 12.7 and 24.7±0.2 degrees two theta.

Another embodiment of the invention provides crystalline ladostigil tartrate (Form J1), characterized by an x-ray diffraction pattern having peaks at 3.5, 6.5, 12.8, 19.3, and 21.1 degrees two theta.

Another embodiment of the invention provides a process for preparing crystalline ladostigil tartrate Form J1 including crystallizing ladostigil tartrate from a solution of ladostigil tartrate in methylenechloride by combining the solution with a C₅ to C₇ saturated hydrocarbon and recovering the crystals.

Another embodiment of the invention provides crystalline ladostigil tartrate (Form K), characterized by an x-ray diffraction pattern having peaks at 6.4, 12.1, 12.8, and 14.6±0.2 degrees two theta.

Another embodiment of the invention provides a process for preparing crystalline ladostigil tartrate Form K including placing Form J1 under 10-70% RH for a sufficient time to obtain the crystalline form.

Another embodiment of the invention provides crystalline ladostigil tartrate (Form L), characterized by an x-ray diffraction pattern having peaks at 4.5, 8.9, 11.4, 14.6, and 18.4±0.2 degrees two theta.

Another embodiment of the invention provides a process for preparing crystalline ladostigil tartrate Form L including maintaining a heterogeneous mixture of crystalline ladostigil tartrate in a solvent selected from the group consisting of CH₂Cl₂, dioxane, THF, and mixtures thereof to obtain the crystalline form and recovering the crystalline form.

Another embodiment of the invention provides solid ladostigil tartrate in amorphous form.

Another embodiment of the invention provides a process for preparing solid ladostigil tartrate in amorphous form including spray drying a solution of ladostigil tartrate in a C₁ to C₄ alcohol or in water.

Another embodiment of the invention provides a process for preparing essentially amorphous ladostigil tartrate including precipitating ladostigil tartrate from a solution of ladostigil tartrate in dimethylene chloride by combining or contacting the solution with a cold solid or liquid having a temperature of at least 20° C. below that of the solution and recovering the amorphous form.

Another embodiment of the invention provides a process for preparing crystalline Form A of ladostigil tartrate including heating ladostigil tartrate Form F or H.

Another embodiment of the invention provides a process for preparing crystalline Form A of ladostigil tartrate including cooling Form A1.

Another embodiment of the invention provides a process for preparing crystalline Form E of ladostigil tartrate including heating ladostigil tartrate Form B or C.

Another embodiment of the invention provides a process for preparing crystalline Form B of ladostigil tartrate including heating ladostigil tartrate Form C.

Another embodiment of the invention provides a process for preparing of ladostigil tartrate Form A including exposing ladostigil tartrate Form A1 to high relative humidity.

Another embodiment of the invention provides crystalline ladostigil tartrate hydrate.

Another embodiment of the invention provides crystalline ladostigil tartrate solvate.

Another embodiment of the invention provides a pharmaceutical composition comprising a therapeutically effective amount of ladostigil tartrate selected from the group consisting of A, B, C, E, F, H, I, J, J1, K, L and amorphous form and a pharmaceutically acceptable carrier.

Another embodiment of the invention provides a process of preparing a pharmaceutical composition comprising the step of combining ladostigil tartrate selected from the group consisting of A, B, C, E, F, H, I, J, J1, K, L and amorphous form, or a solution prepared with one or more of these forms, with a pharmaceutically acceptable carrier.

Another embodiment of the invention provides a method of treating Alzheimer's disease comprising administering to a human subject in need thereof the pharmaceutical composition of the present invention.

Another embodiment of the invention provides a method of treating a mammal in need of inhibition of the acetylcholine esterase enzyme comprising administering the pharmaceutical composition of the present invention to the mammal.

Another embodiment of the invention provides a method of treating a mammal in need of inhibition of the monoamine oxidase type B enzyme comprising administering the pharmaceutical composition of the present invention to the mammal.

### BRIEF DESCRIPTION OF THE DRAWINGS

FIG. 1 illustrates a characteristic x-ray diffraction spectrum of ladostigil tartrate form A1.

FIG. 2 illustrates a characteristic differential scanning calorimetric (DSC) thermogram of ladostigil tartrate form A1.

FIGS. 3-5 illustrate characteristic infrared (IR) spectra of ladostigil tartrate form A1.

FIGS. 6-8 illustrate characteristic Raman spectra of ladostigil tartrate form A1.

FIG. 9 illustrates a characteristic x-ray diffraction spectrum of ladostigil tartrate form A.

FIG. 10 illustrates a characteristic differential scanning calorimetric (DSC) thermogram of ladostigil tartrate form A.

FIGS. 11-13 illustrate characteristic infrared (IR) spectra of ladostigil tartrate form A.

FIGS. 14-16 illustrate characteristic Raman spectra of ladostigil tartrate form A.

FIG. 17 illustrates a characteristic x-ray diffraction spectrum of ladostigil tartrate Form B.

FIG. 18 illustrates a characteristic differential scanning calorimetric (DSC) thermogram of ladostigil tartrate Form B.

FIG. 19 illustrates a characteristic x-ray diffraction spectrum of ladostigil tartrate Form C.

FIG. 20 illustrates a characteristic x-ray diffraction spectrum of ladostigil tartrate Form E.

FIGS. 21-23 illustrate characteristic infrared (IR) spectra of ladostigil tartrate form E.

FIG. 24 illustrates a characteristic x-ray diffraction spectrum of ladostigil tartrate Form F.

FIG. 25 illustrates a characteristic x-ray diffraction spectrum of ladostigil tartrate Form H.

FIG. 26 illustrates a characteristic X-ray diffraction spectrum of ladostigil tartrate Form I.

FIG. 27 illustrates a characteristic X-ray diffraction spectrum of ladostigil tartrate Form J.

FIG. 28 illustrates a characteristic X-Ray diffraction spectrum of ladostigil tartrate Form J1.

FIGS. 29-32 illustrate characteristic FTIR spectra of ladostigil tartrate Form J1.

FIG. 33 illustrates a characteristic DSC thermogram of ladostigil tartrate Form J1.

FIG. 34 illustrates a characteristic X-Ray diffraction spectrum of ladostigil tartrate Form K.

FIG. 35 illustrates a characteristic DSC thermogram of ladostigil tartrate Form K.

FIG. 36 illustrates a characteristic X-Ray diffraction spectrum of ladostigil tartrate Form L.

FIGS. 37-40 illustrate characteristic FTIR spectra of ladostigil tartrate Form L.

FIG. 41 illustrates a characteristic DSC thermogram of ladostigil tartrate Form L.

FIG. 42 illustrates a characteristic X-Ray powder diffractogram of Essentially Amorphous ladostigil tartrate.

FIG. 43 illustrates a characteristic DSC thermogram of Essentially Amorphous ladostigil tartrate.

FIGS. 44-47 illustrate characteristic FTIR spectra of ladostigil tartrate Form F.

FIG. 48 illustrates a characteristic DSC thermogram of ladostigil tartrate Form F.

FIGS. 49-52 illustrate characteristic FTIR spectra of ladostigil tartrate Form E.

FIG. 53 illustrates a characteristic X-Ray powder diffractogram of ladostigil tartrate purely amorphous form.

FIGS. 54-57 illustrate characteristic FTIR spectra of ladostigil tartrate purely amorphous.

FIG. 58 illustrates a DSC thermogram of ladostigil tartrate purely amorphous form.

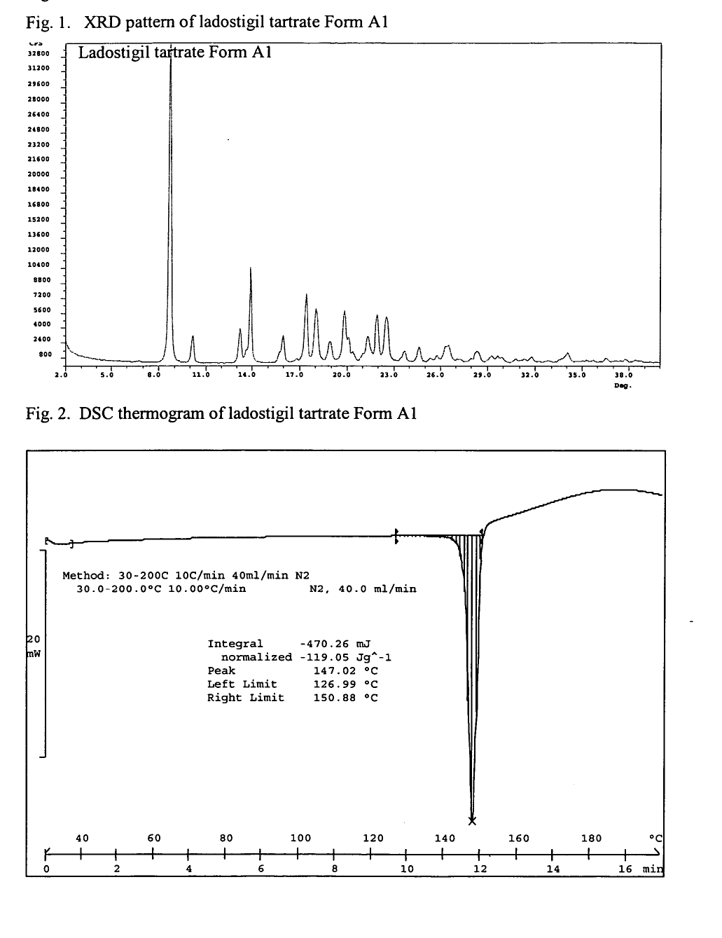

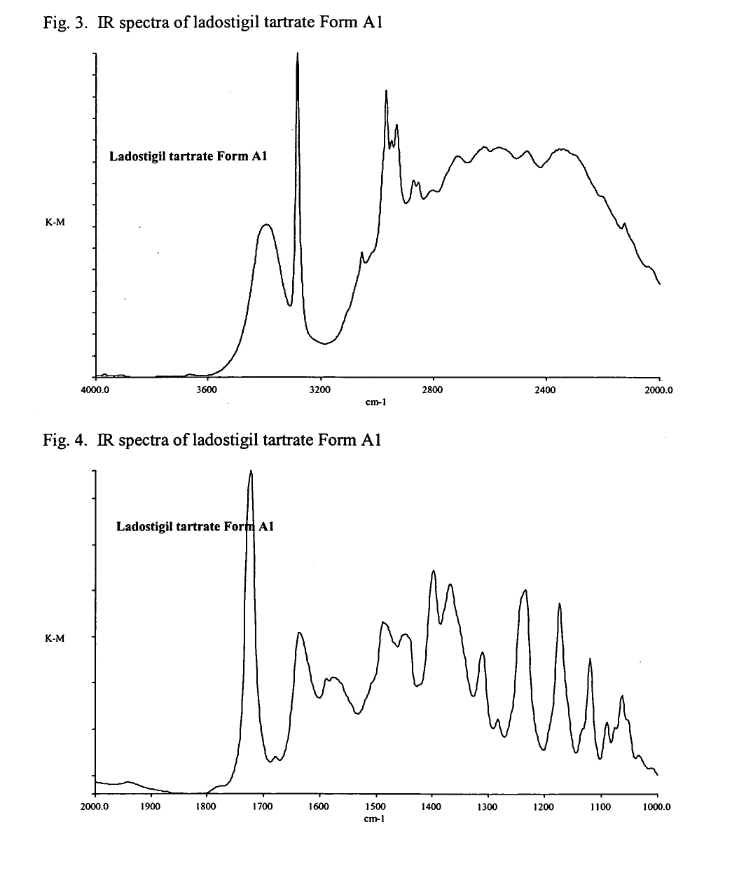

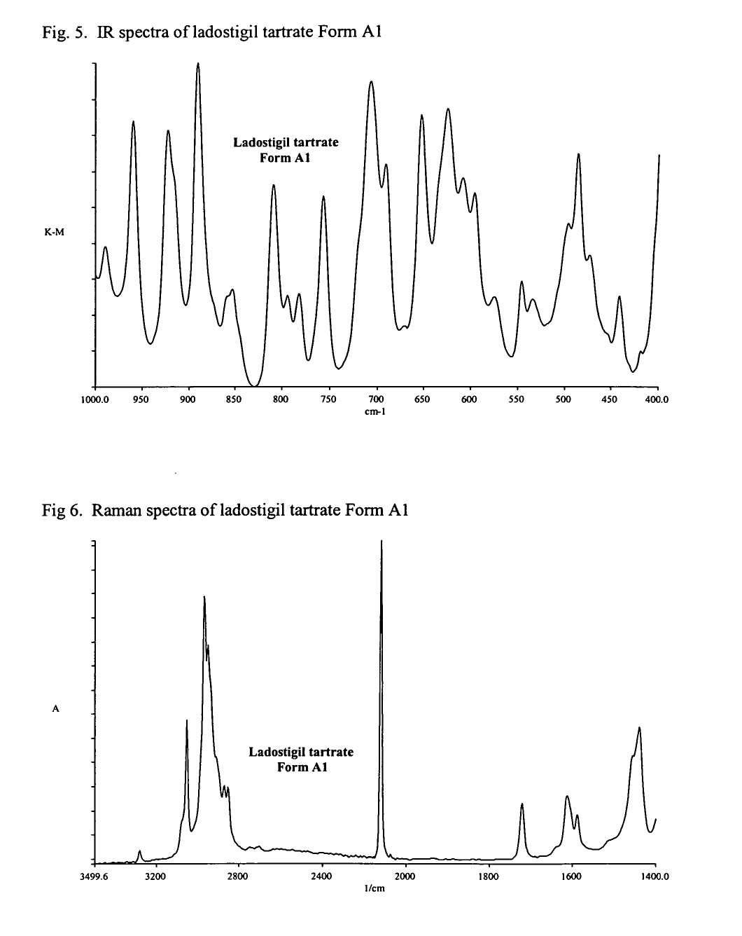

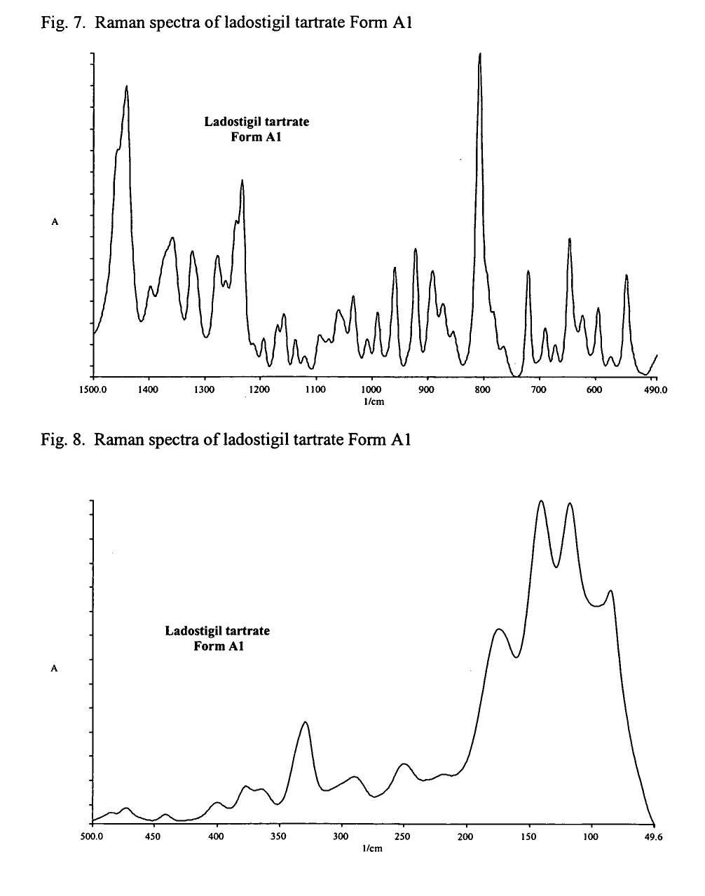

### DETAILED DESCRIPTION OF THE INVENTION

The following is a list of abbreviations used throughout the application:

| | |
|---|---|
| MAO | Monoamine oxidase |
| DSC | Differential scanning calorimetry |
| IR | Infrared |
| TGA | Thermogravimetric analysis |
| MeOH | Methanol |
| MEK | Methyl ethyl ketone |
| FTIR | Fourier transform infrared |
| XRD | X-ray diffraction |
| RH | Relative humidity |
| IPA | Isopropyl alcohol |
| EtOAc | Ethyl acetate |
| EtOH | Ethanol |
| KF | Karl Fischer |

As used herein, the term “vacuum” refers to a pressure below about 100 mmHg.

The invention provides ladostigil tartrate crystalline forms A, B, C, E, F, H, I, J, J1, K, L and ladostigil tartrate amorphous form and processes for their preparation.

The crystalline forms of the invention contain less than about 10%, more preferably less than about 5%, and most preferably less than about 1% of any other crystalline form, especially Form A1, as measured by area percentage XRD.

Form A

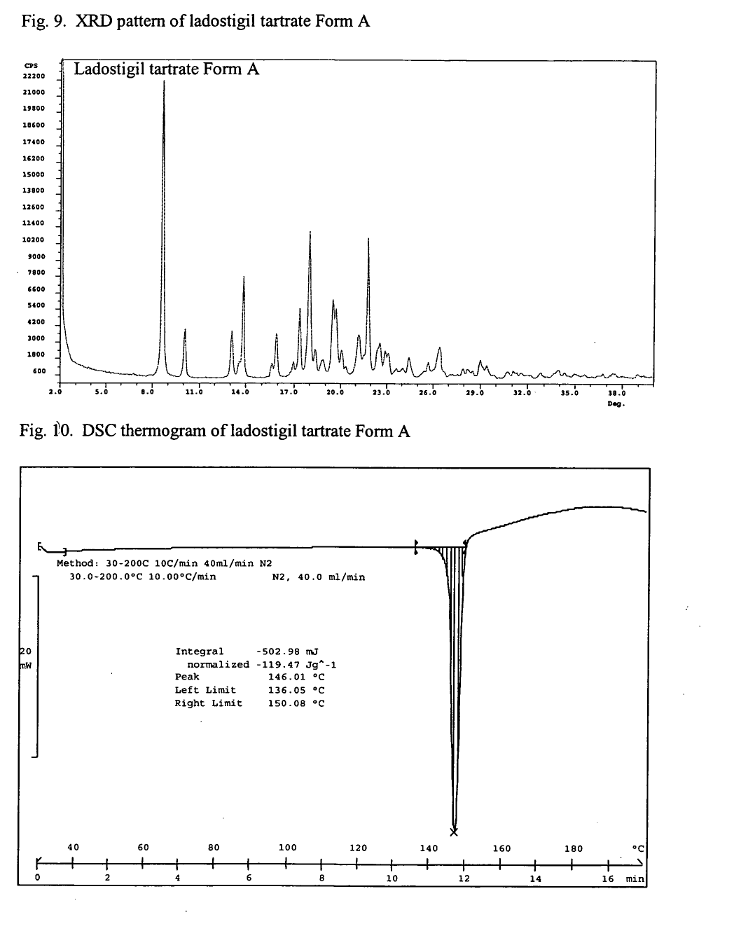

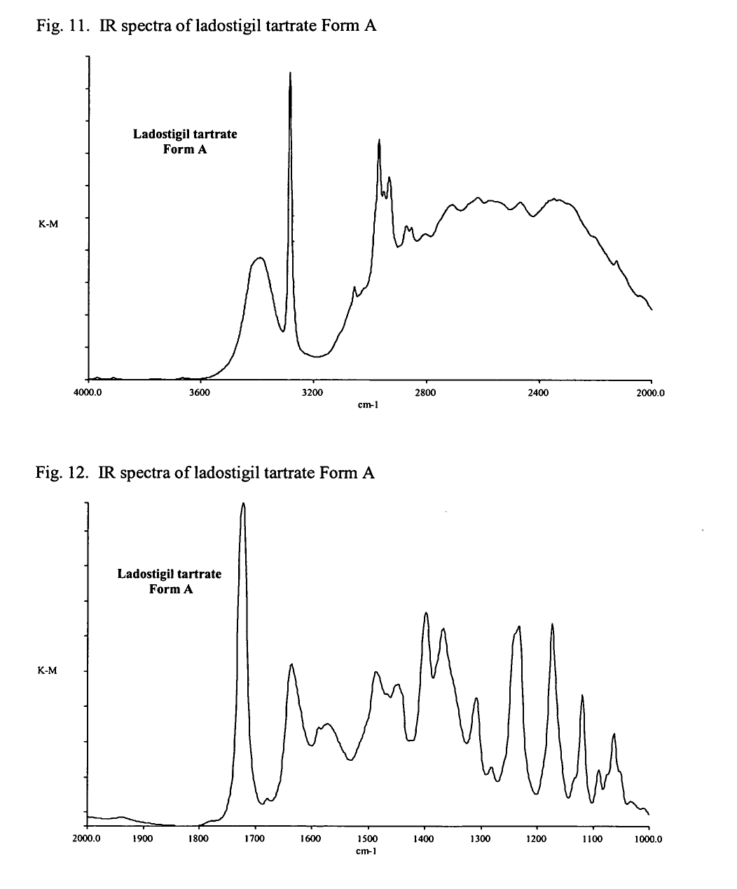

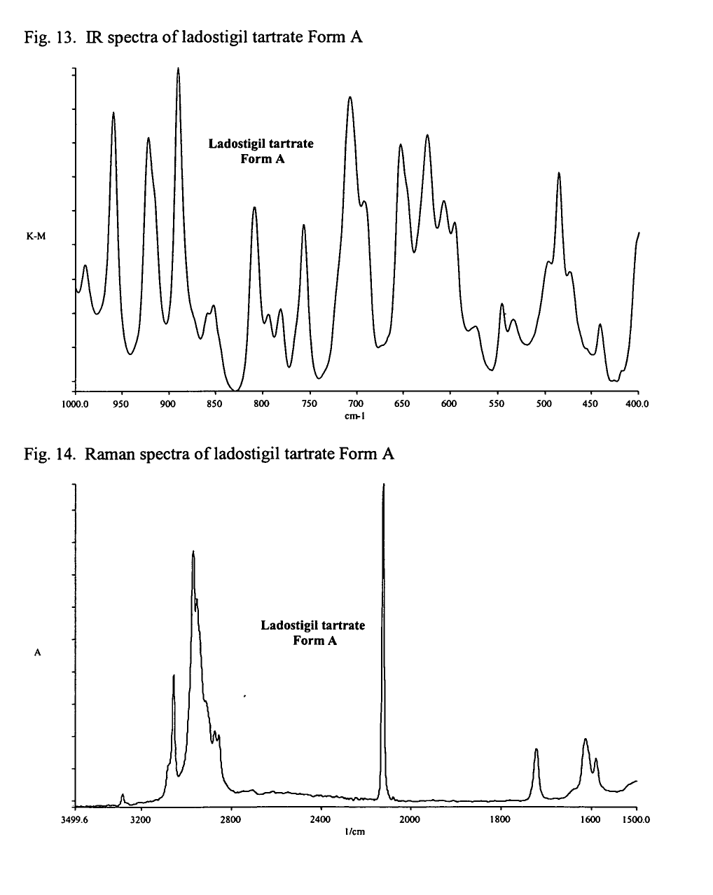

The invention provides for crystalline ladostigil tartrate Form A. Form A is characterized by an XRD pattern having peaks at 17.0, 18.4, 19.5, 22.9, 23.1 degrees 2 theta ±0.2 degrees 2 theta. Form A may be further characterized by an XRD pattern having peaks at 8.7, 10.1, 13.9, 17.5, 18.0, 19.7, 21.8, degrees two theta ±0.2 degrees two-theta. The XRD pattern of crystalline ladostigil tartrate Form A is illustrated in FIG. 9. Crystalline ladostigil tartrate Form A has a DSC thermogram as substantially depicted in FIG. 10, in which about a 120 J/g endothermic peak can be seen at about 145° C. The endotherm corresponds to the melting of the ladostigil tartrate Form A. The water content of the sample is about 0.3% by weight. The loss upon drying by TGA is about 0.3% by weight. The IR spectrum of crystalline ladostigil tartrate Form A is illustrated in FIGS. 11-13 and the Raman spectrum is illustrated in FIGS. 14-16.

Crystalline ladostigil tartrate Form A is anhydrous and non-hygroscopic, and has a stable crystal structure which does not change crystalline form upon exposure to high relative humidity. Form A1 of ladostigil tartrate, on the other hand, transforms to form A upon exposure to high relative humidity. Form A is advantageous in that it is stable at high humidity. Crystalline Form A1 is characterized by an XRD pattern having peaks at 8.7, 13.9, and 17.4±0.2 degrees 2 theta. Form A1 is further characterized by an XRD pattern having peaks at 19.8, 22.0, and 22.5±0.2 degrees 2 theta.

Crystalline ladostigil tartrate Form A may be prepared by the processes disclosed in the examples. For example, Form A may be prepared by crystallization from a solution of ladostigil tartrate in water, tetrahydrofuran, isopropanol, methylene chloride, or mixtures thereof. Preferably, crystallization includes the steps of providing a solution of ladostigil tartrate in water, tetrahydrofuran, isopropanol, methylene chloride, and mixtures thereof, and cooling the solution to crystallize ladostigil tartrate Form A. A solution may be provided by heating ladostigil tartrate in a solvent selected from the group consisting of water, tetrahydrofuran, isopropanol, methylene chloride, and mixtures thereof, to elevated temperature. Preferably, the solution is heated to a temperature close to the boiling point of the solvent. Cooling of the solution is preferably from a temperature of at least about 40° C. to a temperature of below about 20° C. Crystalline ladostigil tartrate Form A may be recovered by conventional techniques in the art, such as filtration.

Due to the thermal stability and anhydrous nature of Form A, the invention also provides other processes of obtaining Form A. For example, in another embodiment of the invention, Form A is obtained by heating other less thermally stable forms. Preferably, the process of the invention includes crystallizing ladostigil tartrate from a solution of ladostigil tartrate in ethanol, acetonitrile, dioxane and mixtures thereof to obtain a wet crystal, heating the wet crystal to obtain crystalline Form A, and recovering the crystalline Form A. Heating is preferably carried out at a temperature of about 40° C. to about 100° C., more preferably about 70° C. to about 90° C.

Form B

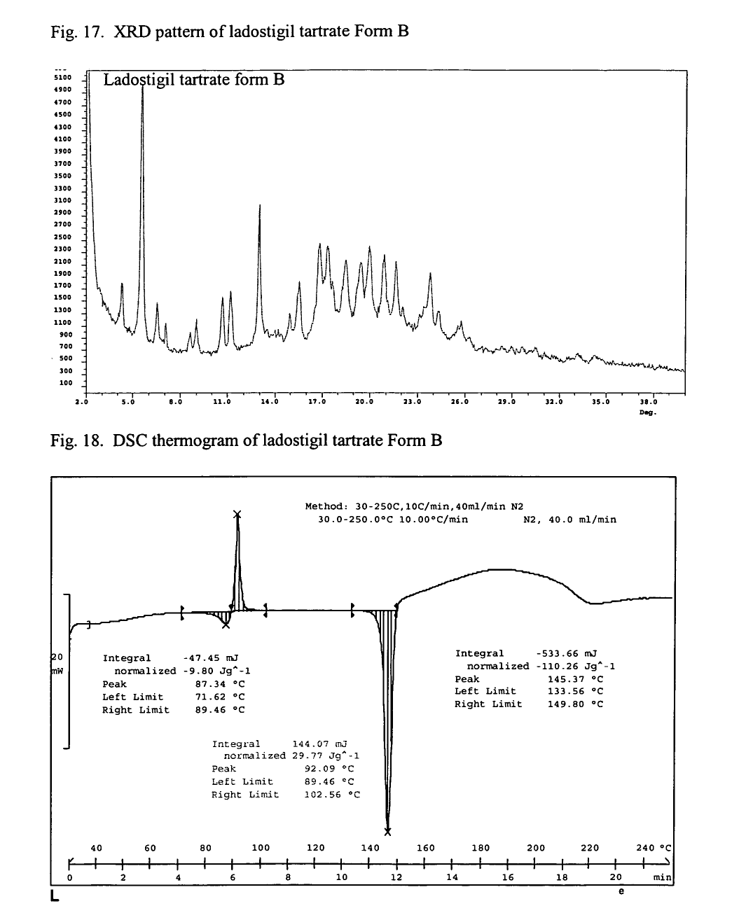

Ladostigil tartrate Form B is characterized by an X-Ray diffraction pattern having peaks at 4.3, 5.6, 13.0, 16.8, 19.9±0.2 degrees 2 theta. Form B is further characterized by an XRD pattern having peaks at 6.5, 10.6, 11.2, 15.5, 20.9, 21.6, 23.8±0.2 degrees 2 theta. Ladostigil tartrate form B has an X-ray powder diffractogram as substantially depicted in FIG. 17. The DSC thermogram of form B is illustrated in FIG. 18. It shows a small endothermic peak followed by an exothermic peak at about 87° C. and 92° C. attributed to the conversion of form B into form A1. These two peaks are followed by the known endothermic peak of Form A1 at about 147° C., followed by an exothermic peak at about 190° C., which correspond respectively to the melting and decomposition of form A1. Form B contains about 1% loss on drying as determined by TGA.

The invention provides a process for preparing Form B by crystallizing ladostigil tartrate Form B by rapid precipitation from a solution of ladostigil tartrate in a C₁ to C₄ alcohol. Rapid precipitation may be carried out by heating a solution of ladostigil tartrate in a C₁ to C₄ alcohol, preferably 2-propanol, followed by rapid cooling of the solution to obtain crystalline ladostigil tartrate Form B. Heating may be carried out at a temperature of at least about 40° C., more preferably at least about 60° C. Rapid cooling may be carried out by combining or contacting the solution with a cold solid or liquid having a temperature of at least 30° C. below that of the solution, to obtain crystalline ladostigil tartrate Form B.

In another embodiment of the invention, rapid precipitation is carried out by combining an anti-solvent with a solution of ladostigil tartrate in a C₁ to C₄ alcohol. Preferably, the anti-solvent is combined with the solution of ladostigil tartrate at one time or in one portion. The anti-solvent is preferably a C₅ to C₇ cyclic or acyclic saturated hydrocarbon, and is more preferably selected from the group consisting of toluene, acetone, ethyl acetate, and dioxane.

The term “anti-solvent” refers to a liquid that, when combined with a solution of ladostigil tartrate in a solvent, induces precipitation of crystalline ladostigil tartrate. The anti-solvent may also be in a binary mixture with the solvent when the solution is prepared. Precipitation of crystalline ladostigil tartrate is induced by the anti-solvent when combination of the solution with the anti-solvent causes crystalline ladostigil tartrate to precipitate from the solution more rapidly or to a greater extent than crystalline ladostigil tartrate precipitates from a solution containing an equal concentration of ladostigil tartrate in the same solvent when the solution is maintained under the same conditions for the same period of time but without adding the anti-solvent. Precipitation can be perceived visually as a clouding of the solution or formation of distinct particles of crystalline ladostigil tartrate suspended in or on the surface of the solution or collected on the walls or at the bottom of the vessel containing the solution.

In the method of the invention, ladostigil tartrate Form B is preferably precipitated by rapid precipitation within about 1 hour of rapid cooling or combination with an anti-solvent.

Form C

Ladostigil tartrate Form C is characterized by an X-Ray diffraction pattern having peaks at ±0.2 degrees 2 theta, as shown in Table 6 below. The DSC thermogram of form C shows small endothermic peaks probably due to the evaporation of solvents from the sample. An analysis of heating of Form C at 50° C. shows that with the evaporation of solvent, form C transforms to form B. These desolvation peaks are followed by an endothermic and exothermic peak at about 87° C. and 92° C., respectively, due to the conversion of Form B into form A1. These two peaks are followed by the known endothermic peak of Form A1 at about 147° C. followed by an exothermic peak at about 190° C. which correspond respectively to the melting and decomposition of form A1.

TGA analysis of the studied form C shows a weight loss step of about 40% w/w due to the removal of solvents. Water content by Karl Fisher analysis is about 2% w/w. From this disparity in the difference of weight loss and water content we deduce that most of the moisture content is due to the presence of solvent.

Form C is an ethanolate, and is prepared from reactions mixtures containing ethanol. Form C may be prepared both from absolute ethanol (less than about 2% water by volume) or from ethanol containing a substantial volume of water, such as about 10% by volume. Form C is generally prepared by crystallizing ladostigil tartrate from a solution of ladostigil tartrate in ethanol. Crystallization may be carried out by providing a solution of ladostigil tartrate in ethanol, heating the solution, cooling of the solution, and recovering crystalline Form C.

A solution of ladostigil tartrate in ethanol may be prepared by dissolving ladostigil tartrate in ethanol, or by dissolving ladostigil base which is then combined with tartaric acid to form ladostigil tartrate. Thus, the starting material may be ladostigil tartrate or ladostigil base, which is then combined with tartaric acid. The solution, in addition to ethanol, may contain up to about 20% by volume co-solvents and/or anti-solvent without changing the polymorphic form obtained. Examples of such co-solvents and anti-solvents include C₃ to C₇ ketones and esters, such as acetone and ethyl acetate; dioxane; C₅ to C₇ hydrocarbons such as toluene; and water. Heating of the solution is preferably to a temperature of about 40° C. to about 60° C. Cooling is preferably to a temperature of about 0° C. to about 25° C. Crystalline Form C may be recovered by conventional methods.

Forms E, F, H, I, and J

Ladostigil tartrate Forms E, F, H, I, and J may be characterized by X-Ray diffraction patterns having peaks as described below in Table 6.

Form E

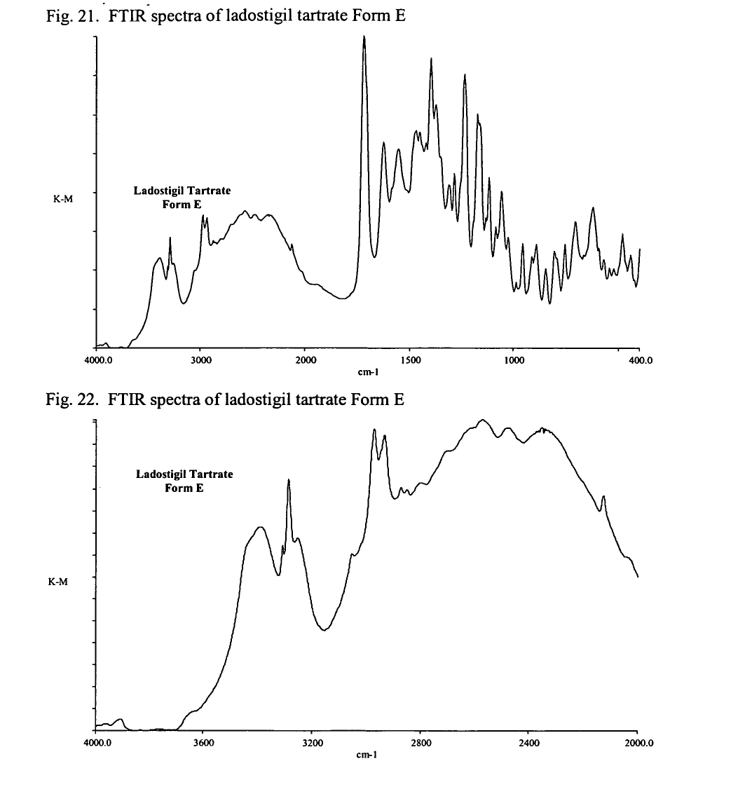

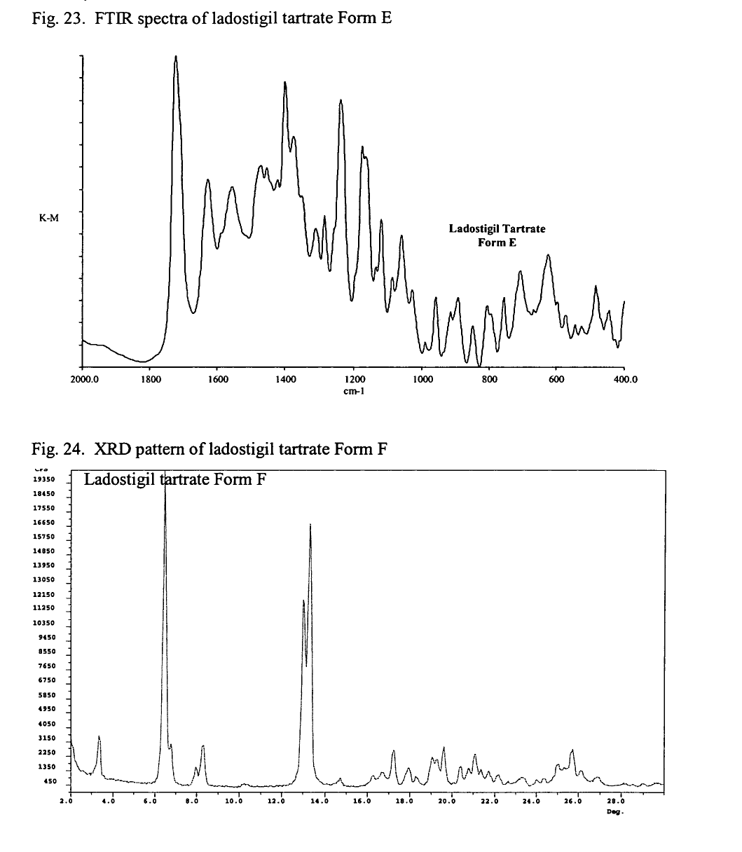

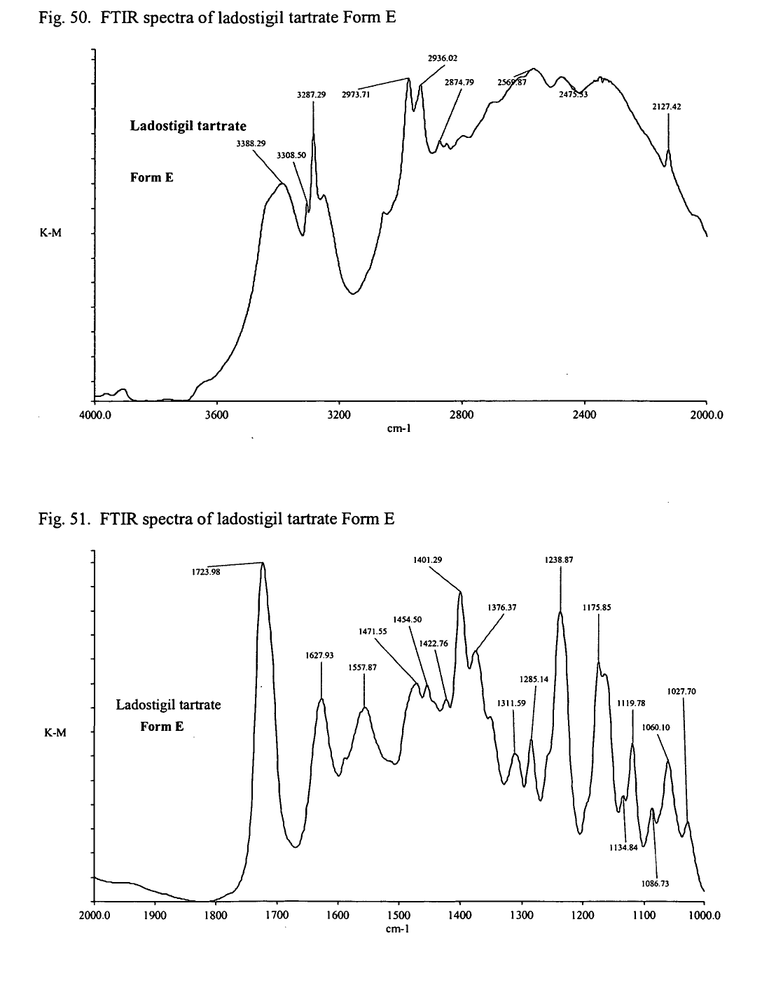

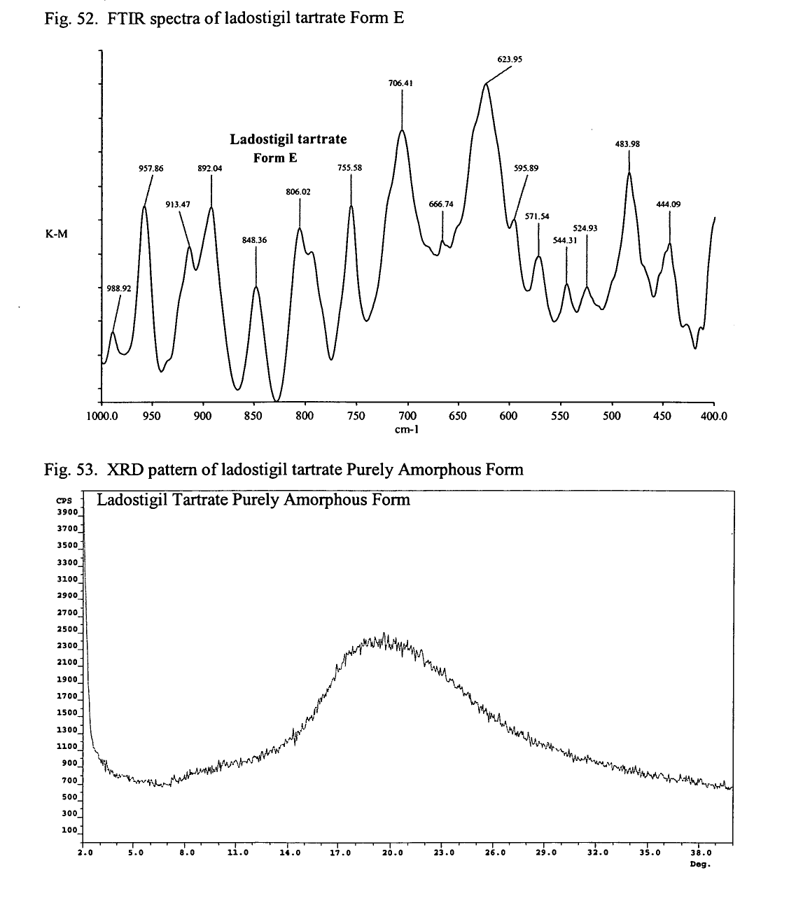

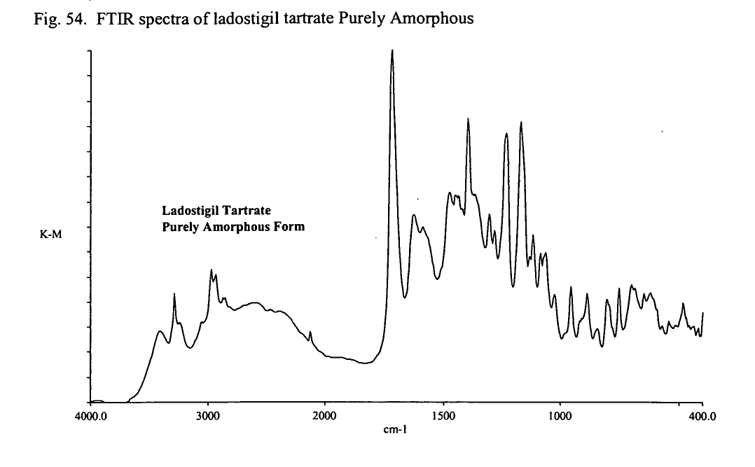

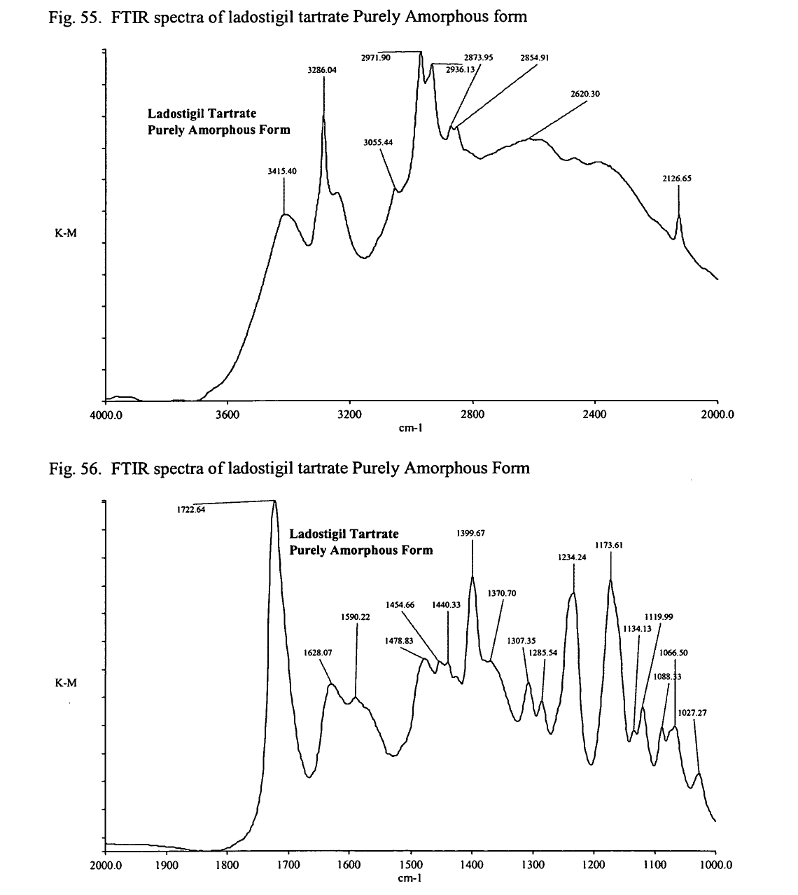

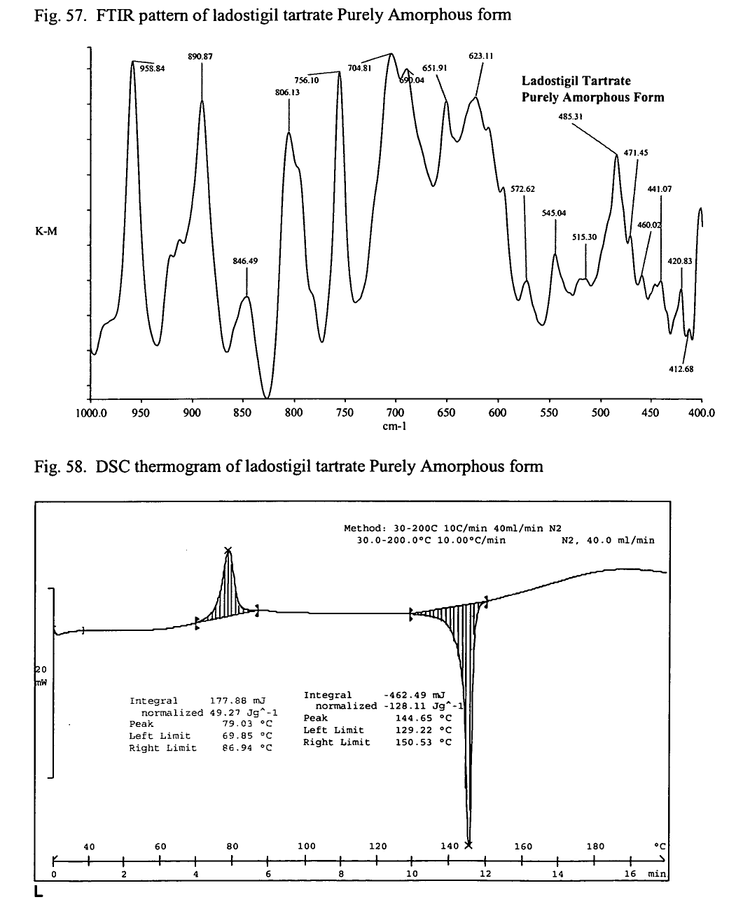

Form E has about 0.7-2% water by weight. Form E may also be characterized by a weight loss measured by TGA of about 0.7-2% by weight. Form E is a hemihydrate. Form E may also be characterized by an FTIR spectrum with characteristic absorption bands at about 3309, 3252, 1628, cm⁻¹, as illustrated in FIGS. 21-23 and 49-50. The term “hydrate” refers to crystalline ladostigil tartrate having one or more water molecules incorporated into the crystalline lattice.

Form E may be prepared by lyophilization from an aqueous solvent, and as such may be highly suitable for pharmaceutical formulations, particularly injectable formulations. In many compositions, the amorphous form is generally isolated by lyophilizing or freeze drying of a solution, entirely skipping any crystallization or isolation step such as complete solvent removal or addition of an anti-solvent. In the case of ladostigil tartrate, we have found that lyophilization can be used to isolate form E. In Drugs and the Pharmaceutical Sciences, Polymorphism in Pharmaceutical Sciences, Vol. 95, the author lists lyophilization as a process utilized to obtain amorphous form of various pharmaceuticals. The authors omit use of lyophilization from processes used to obtain crystalline forms.

In the process of the invention, the aqueous solvent is preferably water or a mixture of water and a C₁ to a C₄ alcohol. The alcohol is preferably a C₁ to a C₄ alcohol and most preferably methanol. High water to solvent ratios are generally preferred. Use of alcohols alone for lyophilization poses a serious health risk. In the most preferred embodiment, the aqueous solvent is water.

The solution is lyophilized according to procedures well known in the art. Lyophilization is a stabilizing process in which a substance is first frozen and then the quantity of the solvent (generally water) is reduced, first by sublimation (referred to as the primary drying process) and then desorption (known as the secondary drying process) to values that will no longer support chemical reactions. One of skill in the art would appreciate that many factors influence the efficiency of lyophilization. These factors include: surface area of sample, eutectic temperature, vacuum, condenser temperature, thickness of the sample, solute concentration and instrument factors. Example 24 provides guidance as to such factors.

In addition to lyophilization, Form E may be obtained by heating crystalline Form C. However, formation of Form E in this process is minimal and in mixture with A1, possibly pointing to a relationship between From E and A1 in regard to thermal transitions.

Form E was placed for 1 week under 0-100% relative humidity at room temperature. Table 1 summarizes the results:

TABLE 1
Hygroscopicity study of Form E

| Relative humidity [%] | Water content by Karl Fisher [%] | Crystal Form |
|---|---|---|
| 0 | 0.7 | E |
| 20 | 0.7 | E |
| 40 | 1.5 | E |
| 60 | 1.8 | E |
| 80 | 0.5 | A1 > E |
| 100 | 0.5 | A1 + A |

Table 1 illustrates that Form E is stable between about 0-60% RH, but between about 80 and 100% RH, Form E transforms to Form A1 and A. Form E is stable at lower humidity and may be used for administration in pharmaceutical compositions. Further, Form E may be used as an intermediate in the preparation of Form A or A1.

Form F

Form F has about 1.3-2.6% water by weight. Form F may also be characterized by a weight loss measured by TGA of about 3% by weight. Form F is a monohydrate. Form F additionally may be characterized by an FTIR spectrum with characteristic absorption bands at about 3425, 3296, 1628, 1403 cm⁻¹. Form F also may be characterized by a DSC thermogram with a broad endothermic peak at 70° C., an exothermic peak at about 90° C., and a final melting endotherm 145° C. with decomposition at 170° C.

Form F is generally prepared by slurry, i.e., a heterogeneous mixture. Slurrying of various crystalline forms of ladostigil tartrate in DMF, acetone, hexane, acetonitrile and mixtures thereof results in Form F. Preferably, form F is prepared by slurry of form A1 in acetonitrile. Transition to Form F may be prolonged, but in example 16c, less than 24 hours was needed to obtain a transition. Preferably the slurrying is carried out for at least about 4 hours.

In addition to slurry, Form F may be prepared by prolonged crystallization from the above solvents. An anti-solvent may be used in the atmosphere to induce crystallization. The anti-solvent is preferably a saturated C₅ to C₇ hydrocarbon, and more preferably is hexane, cyclohexane, heptane or cycloheptane. Crystalline Form F may be recovered by conventional techniques, such as filtration.

Form F was placed for 14 days under 0-100% relative humidity at room temperature. Table 2 summarizes the results:

TABLE 2
Hygroscopicity study of Form F

| | | Loss on | |
|---|---|---|---|
| Relative | Water content by | Dry by TGA | |
| humidity [%] | Karl Fisher [%] | [%] | Crystal Form |
| 0 | 1.3 | Not measured | Form F |
| 20 | 2.2 | 3.0 | Form F |
| 40 | 2.6 | 2.9 | Form F |
| 60 | 0.3 | 0.3 | Form A1 + ˜10% E |
| 80 | 0.3 | 0.2 | Form A1 + ˜20% A |
| 100 | 0.3 | 0.3 | Form A + ˜20% A |

Table 2 illustrates that Form F is stable of about 0-40% RH, but at about 60 and 100% RH, Form F transforms to Form E, A1 and A. Form F is stable at lower humidity and may be used for administration in pharmaceutical compositions. Further, Form F may be used as an intermediate in the preparation of Form A or A1.

Form H

Crystalline ladostigil tartrate Form H has about 0.6% water by weight. Form H may also be characterized by a weight loss measured by TGA of about 6% by weight.

Form H of the invention is a dioxane solvate. The dioxane solvate may be prepared from various mixtures containing dioxane. The term “solvate” refers to crystalline ladostigil tartrate having one or more solvent molecules incorporated into the crystalline lattice.

In one embodiment, the invention provides a process for preparing crystalline Form H by crystallizing Form H from a solution of ladostigil tartrate in dioxane. In one preferred embodiment, crystallizing includes heating the solution of ladostigil tartrate in dioxane, followed by cooling, and recovering crystalline Form H. The solution is preferably heated to a temperature of at least about 40° C., followed by cooling to a temperature below about 20° C. Crystalline Form H may be recovered by conventional techniques, such as by filtration.

Form H was placed for 14 days under 0-100% relative humidity at room temperature. Table 3 summarizes the results:

TABLE 3
Hygroscopicity study of Form H

| | | Loss on | |
|---|---|---|---|
| Relative | Water content by | Dry by TGA | |
| humidity [%] | Karl Fisher [%] | [%] | Crystal Form |
| 0 | 0.42 | 6.1 | Form H |
| 20 | 0.7 | 5.2 | Form F |
| 40 | 1.4 | 2.7 | Form L |
| 60 | 0.3 | 0.5 | Form A1 + ˜30% E |
| 80 | 0.2 | 0.2 | Form A1 + ˜20% A |
| 100 | 0.3 | 0.2 | Form A + ˜30% A1 |

Table 3 illustrates that Form H transforms to Form F at about 20% RH, to Form L at about 40% RH and to Form E, A1 and A at about 60-100% RH. Form H may be used as an intermediate in preparation of Forms A or A1.

Form I

The invention also provides ladostigil tartrate acetonitrile solvate. The acetonitrile solvate, Form I, may be prepared from various mixtures containing acetonitrile.

In one embodiment, the invention provides a process for preparing crystalline Form I by maintaining a heterogeneous mixture of ladostigil tartrate in acetonitrile to obtain Form I. Preferably, the process comprises slurrying ladostigil tartrate in acetonitrile. Preferably, wet ladostigil tartrate crystallized from ethanol is slurried in acetonitrile to obtain Form I. The volume of acetonitrile relative to the weight of the wet ladostigil tartrate is preferably at least about 4 L/Kg, more preferably at least about 6 L/Kg. After transformation to Form I, the crystalline form may be recovered by conventional techniques.

Form J

The invention also provides a process for preparing crystalline Form J by drying Form I. Preferably, wet Form I obtained from slurry in acetonitrile is dried to obtain Form J. The heating is preferably carried out at a temperature of about 75° C. to about 85° C., more preferably about 80° C. Preferably the heating is carried out under vacuum.

Form J1

The invention also provides crystalline ladostigil Tartrate Form J1. Form J1 has about 1% water by weight, and may also be characterized by a weight loss measured by TGA of about 10% by weight. Form J1 is a solvate of CH₂Cl₂. Crystalline Form J1 may be further characterized by an X-ray powder diffraction pattern having peaks at 3.5, 6.5, 12.8, 19.3, 21.1 degrees two-theta, ±0.2 degrees two-theta. Form J1 may be characterized further by an X-ray powder diffraction having peaks at 8.0, 17.3, 18.0, 21.5, 24.7 degrees two-theta, ±0.2 degrees two-theta. Form J1 may be characterized by an FTIR spectrum with characteristic absorption bands at about 3421, 1721, 1628, 1561, 1374 cm⁻¹. Form J1 also may be characterized by a DSC thermogram with characteristic endothermic peaks at about 100° C. and 150° C. and decomposition at about 170° C.

Being a solvate of methylenechloride, Form J1 may generally be prepared from reaction mixtures containing methylenechloride. In one embodiment, Form J1 is crystallized from a solution of ladostigil tartrate in methylenechloride. A saturated hydrocarbon atmosphere as described above may be used as an anti-solvent to cause slow crystallization over time.

Form J1 was placed for 9 days under 0-100% relative humidity at room temperature. Table 4 summarizes the results:

TABLE 4
Hygroscopicity study of Form J1

| | | Loss on | |
|---|---|---|---|
| Relative | Water content by | Dry by TGA | |
| humidity [%] | Karl Fisher [%] | [%] | Crystal Form |
| 0 | 0.1 | 5.4 | Form J1 |
| 20 | | | Form K + ˜20% J1 |
| 40 | 0.2 | 0.8 | Form K |
| 60 | 0.2 | 0.5 | Form K |
| 80 | | | Form A1 + ˜20% A |
| 100 | 0.0 | 0.1 | Form A + ˜30% A1 |

Table 4 illustrates that Form H transforms partly to Form K at 20% RH, to Form K at 40-60% RH and to Form A1 and A at 80-100% RH. Form K is stable at 20-60% RH, and may be used for administration in pharmaceutical compositions. Further, Form J1 may be used as an intermediate in preparation of Form A or A1.

Form K

One embodiment of the invention provides crystalline ladostigil tartrate Form K. Form K has about 0.2% water by weight, and may also be characterized by a weight loss measured by TGA of about 0.5-0.8% by weight. The invention also provides a process for preparing crystalline Form K by exposing crystals of Form J1 to a humid atmosphere to obtain Form K. Preferably, the humid atmostphere has about 10% to about 70% RH.

Form L

One embodiment of the invention provides crystalline ladostigil Tartrate Form L. Ladostigil tartrate Form L has about 0.6-1.4% water by weight, and may also be characterized by a weight loss measured by TGA of about 2.7-13.8% by weight. Form L may be obtained as a solvate of CH₂Cl₂, dioxane or THF. Crystalline Form L may be identified by an X-ray powder diffraction pattern having peaks at 4.5, 8.9, 11.4, 14.6, 18.4±0.2 degrees two-theta. Form L crystal may be identified further by X-ray powder diffraction peaks at 13.4, 14.1, 16.6, 19.5, 20.7 degrees two-theta, ±0.2 degrees two-theta. Form L may be characterized by an FTIR spectrum with characteristic absorption bands at about 2972, 1722, 1628 cm⁻¹. Form L also may be characterized by a DSC thermogram with endothermic peak at about 90° C., an exothermic peak at about 95° C. which relates to recrystallization and a final melting endotherm at about 145° C. and decomposition at 170° C.

One embodiment of the invention provides a process for preparing Form L by maintaining a heterogeneous mixture of crystalline ladostigil tartrate in a solvent selected from the group consisting of CH₂Cl₂, dioxane, THF and mixtures thereof to obtain the crystalline form and recovering the crystalline form. Preferably, Form L is prepared by slurrying a crystalline form of ladostigil tartrate in CH₂Cl₂, dioxane or THF to obtain Form L. The slurry process may be carried out for about 24 hours. The starting crystalline form may be any crystalline form of ladostigil tartrate, but is preferably Form A1. The resulting crystalline Form L may be recovered by conventional techniques.

Essentially Amorphous Form

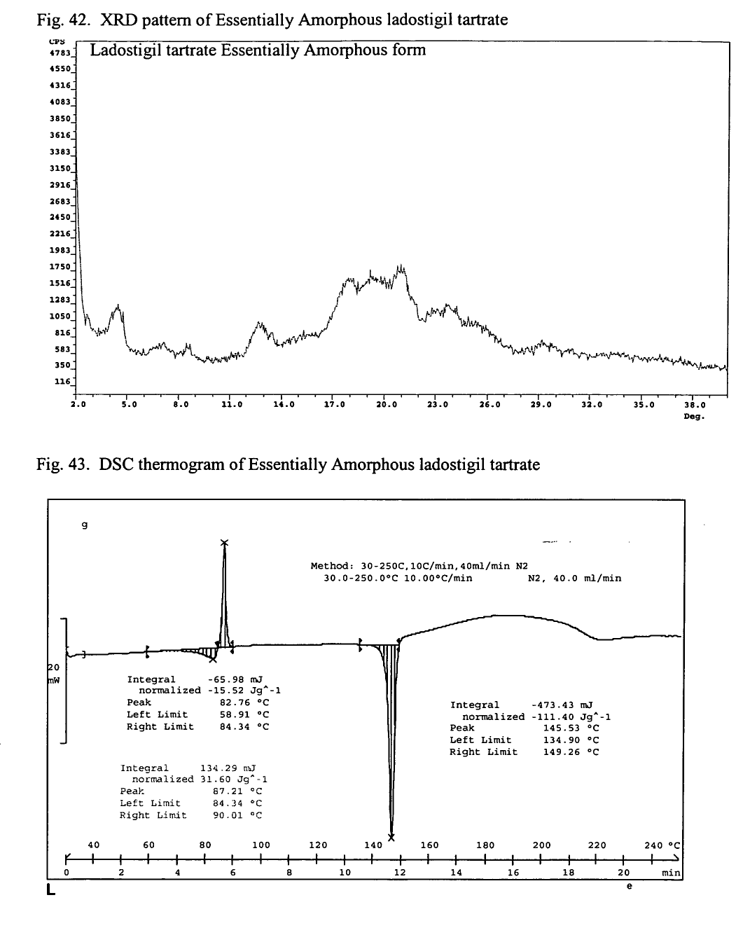

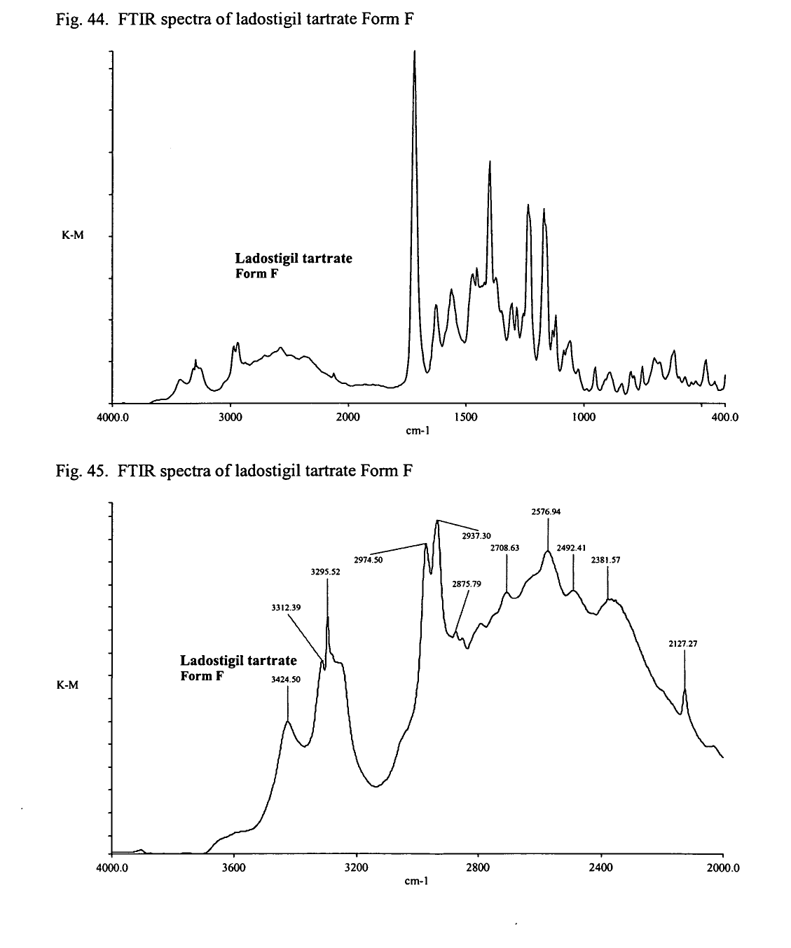

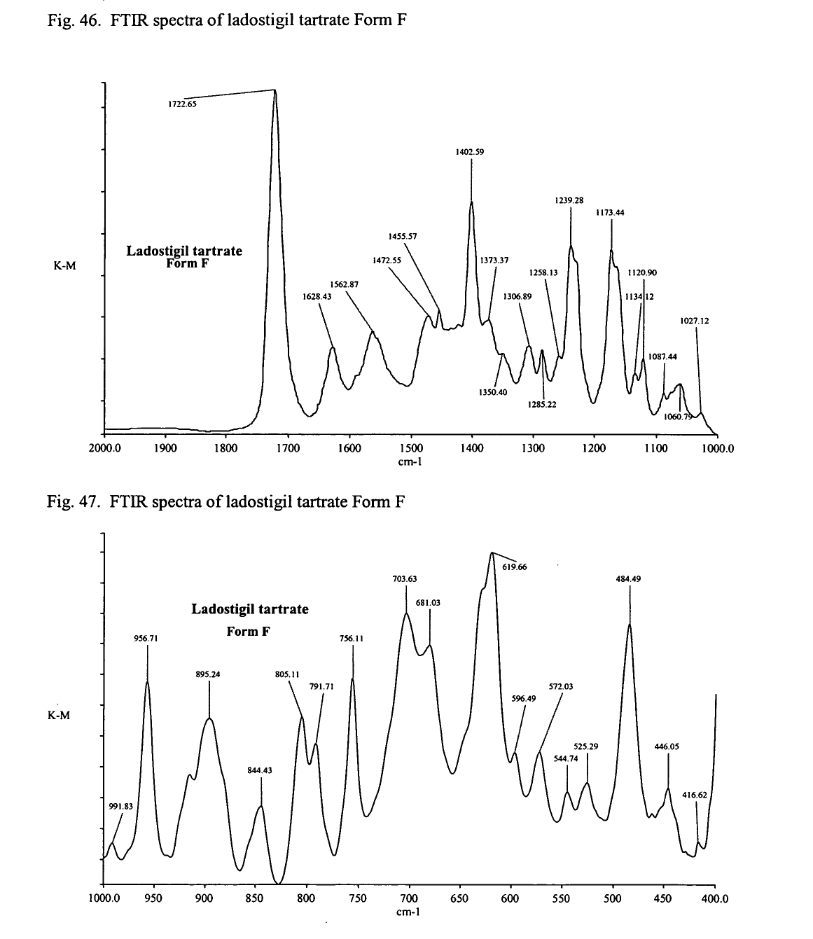

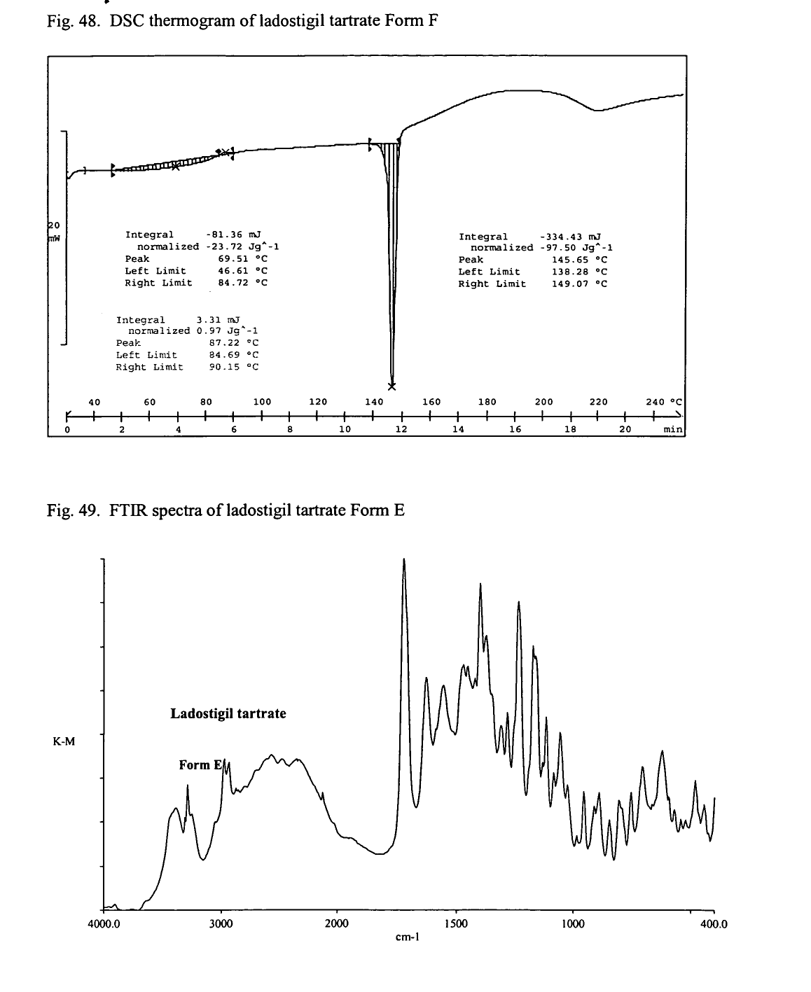

One embodiment of the invention provides Essentially Amorphous Form of ladostigil tartrate. Essentially amorphous form has about 10% to about 30% crystallinity, preferably about 20% crystallinity. Essentially Amorphous form has about 2% water by weight. Essentially Amorphous Form may also be characterized by a weight loss measured by TGA of about 5% by weight. Essentially Amorphous Form may be characterized by a broad X-ray powder diffraction pattern as illustrated in FIG. 42. Essentially Amorphous Form may be characterized by a DSC thermogram with an endothermic peak at ˜80° C., and an exothermic peak at about 87° C. which relates to crystallization, and a final melting endotherm at 145° C. and decomposition at about 170° C. The DSC thermogram for Essentially Amorphous Form is substantially depicted in FIG. 43.

One embodiment of the invention provides a process for preparing ladostigil tartrate essentially amorphous by rapid precipitation of essentially amorphous form from a solution of ladostigil tartrate in methylene chloride. The precipitation is carried out rapidly to avoid formation of crystalline ladostigil tartrate. In one embodiment, rapid precipitation includes providing a solution of ladostigil tartrate in methylene chloride at room temperature, contacting the solution with a cold surface to obtain essentially amorphous ladostigil tartrate, and recovering essentially amorphous form. Preferably, contacting the solution with a cold surface is carried out by adding the solution to a cold surface. The cold surface preferably has a temperature below about 0° C. The solution may optionally be heated before contacting with the cold surface. Essentially amorphous form may be recovered by techniques known in the art.

Purely Amorphous Form

One embodiment of the invention provides Purely Amorphous Form of ladostigil tartrae. The purely amorphous form of the invention preferably contains less than 10% crystalline form, more preferably less than 1%, and most preferably less than 1%, or undetectable amounts of crystalline ladostigil tartrate, as measured by area percentage XRD.

Purely Amorphous Form may be characterized by a broad X-ray powder diffraction pattern. The purity of our amorphous form is apparent from the broad halo pattern in the XRD, which is missing peaks associated with the presence of crystals. Purely Amorphous Form may be characterized by an FTIR spectrum with characteristic absorption bands at about 3415, 1628, 1478, 705 cm-1. Purely Amorphous Form may be characterized by a DSC thermogram with an exothermic peak at about 80° C., which corresponds to crystallization to Form A1, and an endothermic peak at about 145° C. which corresponds to the melting of Form A1. The broad exothermic peak at about 170° C. corresponds to decomposition. Purely amorphous form may be useful in preparing pharmaceutical compositions as the batches of purely amorphous form will more likely have consistent polymorphic characteristics. Consistent polymorphic characteristics will further result in a more uniform bioavailability and stability from batch to batch.

In another aspect, the invention provides a process for preparing purely amorphous ladostigil tartrate including spray drying a solution of ladostigil tartrate in a C₁ to C₄ alcohol, water, or a mixture thereof. According to Remington: The Science and Practice of Pharmacy, 19th Ed., vol. II, pg. 1627, spray drying consists of bringing together a highly dispersed liquid and a sufficient volume of hot air to produce evaporation and drying of the liquid droplets. A typical spray-drying apparatus includes a drying chamber, atomizing means for atomizing a solvent-containing feed into the drying chamber, a source of heated drying gas that flows into the drying chamber to remove solvent from the atomized-solvent-containing feed and product collection means located downstream of the drying chamber. Examples of such apparatus include Niro Models PSD-1, PSD-2 and PSD-4 (Niro A/S, Soeborg, Denmark). Modifications to the spray drying technique is disclosed in WO 03/063821 and WO 03/063,822.

In the process of the invention, a solution of ladostigil tartrate in water, a C₁ to C₄ alcohol, or mixtures thereof, is atomized into a chamber with hot air. The solvent is preferably water or methanol. The hot air is preferably nitrogen. The resulting dry purely amorphous form may be recovered by conventional techniques.

Purely Amorphous Form was placed for 8 days under 0-100% relative humidity at room temperature. Table 5 summarizes the results:

TABLE 5
Hygroscopicity study of Purely Amorphous Form

| | Water | Loss on | |
|---|---|---|---|
| Relative | content by | Dry by TGA | |
| humidity [%] | Karl Fisher [%] | [%] | Crystal Form |
| As is | 2.2 | 2.2 | Amorphous |
| 0 | | 0.5 | Amorphous |
| 20 | | 0.8 | Amorphous |
| 40 | | 1.2 | Amorphous |
| 60 | | 2.2 | Amorphous |
| 80 | | 0.3 | Form A1 |
| 100 | | 0.4 | Form A |

Table 1e illustrates that amorphous Form is stable between about 0-60% RH, but at about 60-80% RH purely amorphous form converts to Form A1 and at about 80-100% RH amorphous Form transforms to Form A. Purely amorphous Form is stable at lower humidity, and may be used for administration in pharmaceutical compositions. Further, amorphous form may be used as an intermediate in preparation of Form A or A1.

TABLE 6
X-Ray Diffraction Peaks for crystalline ladostigil tartrate forms
The following table shows peaks obtained by X-Ray diffraction
for crystalline forms of ladostigil tartrate. The most characteristic
peaks for each form are showed in bold:

| Form E | Form F | Form H | Form I | Form J |
|---|---|---|---|---|
| 4.9 | 3.3 | 4.4 | 6.5 | 6.3 |
| 8.7 | 6.4 | 8.5 | 12.0 | 7.9 |
| 12.0 | 8.3 | 10.5 | 13.0 | 12.2 |
| 13.6 | 13.0 | 11.0 | 13.3 | 12.7 |
| 17.5 | 13.3 | 15.6 | 15.9 | 16.0 |
| 18.9 | 17.2 | 16.0 | 18.6 | 19.1 |
| 20.2 | 19.6 | 16.8 | 19.6 | 20.3 |
| 21.8 | 21.0 | 17.7 | 22.6 | 22.0 |
| | | 18.6 | 25.7 | 24.7 |
| | | 21.2 | | 24.9 |
| | | | | 25.6 |

TABLE 7
Main IR peaks for ladostigil tartrate crystal forms A1, A, B, and C
The most characteristic peaks are marked with an asterisk.

| Form A and A1 | Form B | Form C |
|---|---|---|
| 3388.0* | | 3901.4* |
| 3284.9* | 3290.5* | 3309.4* |
| 3056.1 | | |
| 2970.0* | 2973.5 | 2974.3 |
| 2953.2 | | |
| 2934.0 | 2938.3 | 2936.8 |
| 2873.1 | | |
| 2855.4 | | |
| 2805.3 | | |
| 2711.5 | | 2704.7 |
| 2620.8 | 2576.0 | |
| 2471.0 | | |
| 2351.9 | | |
| 2126.5 | | 2126.5 |
| 1722.8* | 1716.7 | 1716.8 |
| 1636.3* | 1626.8* | 1565.4 |
| 1487.7* | 1562.9 | 1476.0 |
| 1447.2* | 1475.3 | |
| 1398.5 | 1454.2 | 1401.4 |
| 1368.4* | 1401.6 | 1338.1* |
| 1309.0* | 1306.1 | 1305.6 |
| 1233.6* | 1285.6 | 1287.0 |
| 1174.3* | 1238.7* | 1261.0* |
| 1119.8 | 1166.1* | 1238.8 |
| 1090.0 | 1121.0 | 1169.2* |
| 1063.4 | 1087.2 | 1134.9* |
| 989.3* | 1064.0 | 1076.7* |
| 959.2 | 1027.7 | 1067.4* |
| 922.0* | 957.2 | 1027.5 |
| 890.2* | 892.9 | 957.6 |
| 852.4* | 844.2 | 903.3* |
| 808.9* | 804.5 | 843.4 |
| 794.1 | | 803.6 |
| 781.3* | | 790.9* |
| 757.0 | 755.6 | 755.6 |
| 707.7* | 700.9* | |
| 692.3* | | 680.5* |
| 653.6* | | |
| 624.8* | 632.4* | 619.9* |
| 607.1* | 617.4* | |
| 595.8 | 595.8 | 595.8 |
| 573.5 | | 571.4 |
| 545.6 | | |
| 533.6* | | 524.1 |
| 485.8 | 485.2 | 485.2 |
| 473.6 | | 446.5 |
| 441.0 | | 416.7 |

The starting material used in the processes of the invention may be any crystalline or amorphous form of ladostigil tartrate, including various solvates and hydrates. With crystallization processes, the crystalline form of the starting material does not usually affect the final result. With slurry, the final product may very depending on the starting material. One of skill in the art would appreciate the manipulation of the starting material within skill in the art to obtain a desirable form with slurry. The invention is not limited to the starting form used for slurry unless such form is essential for obtaining another form.

Many processes of the invention involve crystallization out of a particular solvent, i.e., obtaining a solid material from a solution. One skilled in the art would appreciate that the conditions concerning crystallization may be modified without affecting the form of the polymorph obtained. For example, when mixing ladostigil tartrate in a solvent to form a solution, warming of the mixture may be necessary to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization.

The conditions may also be changed to induce precipitation. A preferred way of inducing precipitation is to reduce the solubility of the solvent. The solubility of the solvent may be reduced, for example, by cooling the solvent.

In one embodiment, an anti-solvent is added to a solution to decrease its solubility for a particular compound, thus resulting in precipitation. Another way of accelerating crystallization is by seeding with a crystal of the product or scratching the inner surface of the crystallization vessel with a glass rod. Other times, crystallization may occur spontaneously without any inducement. The present invention encompasses both embodiments where crystallization of a particular form of nateglinide occurs spontaneously or is induced/accelerated, unless if such inducement is critical for obtaining a particular form.

A solid may be recovered from a reaction mixture in a routine fashion such as by filtration, centrifugation or decanting.

Ladostigil tartrate of defined particle size may be produced by known methods of particle size reduction starting with crystals, powder aggregates and course powder of the new crystalline forms of tegaserod maleate. The principal operations of conventional size reduction are milling of a feedstock material and sorting of the milled material by size.

A fluid energy mill, or micronizer, is an especially preferred type of mill for its ability to produce particles of small size in a narrow size distribution. As those skilled in the art are aware, micronizers use the kinetic energy of collision between particles suspended in a rapidly moving fluid stream to cleave the particles. An air jet mill is a preferred fluid energy mill. The suspended particles are injected under pressure into a recirculating particle stream. Smaller particles are carried aloft inside the mill and swept into a vent connected to a particle size classifier such as a cyclone. The feedstock should first be milled to about 150 to 850 μm which may be done using a conventional ball, roller, or hammer mill. One of skill in the art would appreciate that some crystalline forms may undergo a transition to another form during particle size reduction.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

The invention also provides a pharmaceutical composition comprising a therapeutically effective amount of ladostigil tartrate selected from the group consisting of A, B, C, E, F, H, I, J, J1, K, L, amorphous form and mixtures thereof, and a pharmaceutically acceptable carrier.

The invention also provides a process for preparing pharmaceutical compositions containing ladostigil tartrate selected from the group consisting of A, B, C, E, F, H, I, J, J1, K, L, amorphous form and mixtures thereof, comprising admixing a pharmaceutically acceptable excipient with ladostigil tartrate selected from the group consisting of A, B, C, E, F, H, I, J, J1, K, L, amorphous form and mixtures thereof. The invention also provides a method of treating Alzheimer's disease in a mammal, such as a human, comprising administering to the mammal a pharmaceutically acceptable amount of a pharmaceutical composition containing or prepared from ladostigil tartrate selected from the group consisting of A, B, C, E, F, H, I, J, J1, K, L, amorphous form and mixtures thereof.

The pharmaceutical composition may contain only a single form of ladostigil tartrate, or a mixture of various forms of ladostigil tartrate, with or without amorphous form. In addition to the active ingredient(s), the pharmaceutical compositions of the invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dixoide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the invention, the active ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the invention may comprise any of the aforementioned blends and granulates that were described with reference to tableting, however, they are not subjected to a final tableting step.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The dosage used is preferably from about 0.5 mg to about 500 mg of ladostigil tartrate, more preferably about 20 to about 100 mg.

Experimental Procedures

X-Ray powder diffraction data were obtained by method known in the art using a SCINTAG powder X-Ray diffractometer model X'TRA equipped with a solid state detector. Copper radiation of 1.5418 Å was used. A round aluminum sample holder with round zero background quartz plate, with a cavity of 25(diameter)*0.5(depth) mm was used.

The following parameters were used:

Range: 2-40 degrees two-theta.

Scan mode: Continuous scan

Step size: 0.05 deg.

Rate: 5 deg./min.

DSC analysis was performed using a Mettler 821 Star. The weight of the samples was about 5 mg. The samples were scanned at a rate of 10° C./min from 30° C. to 320° C. The oven was constantly purged with nitrogen gas at a flow rate of 40 ml/min. Standard 40 μl aluminum crucibles covered by lids with 3 holes were used.

TGA analysis was performed using a Mettler M3 meter. The weight of the samples was about 10 mg. The samples were scanned at a rate of 10° C./min from 25° C. to 200° C. The oven was constantly purged with nitrogen gas at a flow rate of 40 ml/min. Standard 70 μl alumina crucibles covered by lids with 1 hole were used.

IR analysis was performed using a Perkin Elmer “Spectrum One” FT-IR spectrometer in DRIFT mode. The samples in the 4000-400 cm⁻¹ interval were scanned 16 times with 4.0 cm⁻¹ resolution.

Raman spectroscopy was performed using a Bruker RFS-100/S Raman spectrometer. The samples in the 3500-50 cm⁻¹ interval were scanned 100 times with 4.0 cm⁻¹ resolution. Other parameters were set as follows:

| | |
|---|---|
| Aperture Setting | 10.0 mm |
| Low Pass Filter 16; | 1 KHz |
| Source Setting Laser; 9394.0 cm−1; | 1600 mW |
| Raman Laser Power(mW) | 500 |
| Scanner Velocity 5.0 | 4 KHz |

Spray-Drying Instrument
Mini Spray Dryer B-290 with Inert Loop B-295, Buchi, Switzerland.

The water content of ladostigil tartrate was measured by methods known in the art, such Karl Fischer analysis.

The examples and preparations provided below further illustrate and exemplify the compounds of the present invention and methods of preparing such compounds. It is to be understood that the scope of the present invention is not limited in any way by the scope of the following examples and preparations.

### EXAMPLES

Polymorph A

### Example 1

### Re-Crystallization of Ladostigil Tartrate in 5 Volumes Ethanol

Ladostigil tartrate (50 g) is dissolved in ethanol (250 ml, 5 vol.) by heating the solution to 55-62° C. The mixture was cooled to 0-5° C. gradually for 4.5 hours and was maintained at the same temperature for 2.5 hours. The product was collected by filtration and washed with 30 ml cold ethanol.

The material was dried at 60° C. under vacuum for 14 hours, giving polymorph A.

### Example 2

### Re-Crystallization of Ladostigil Tartrate in 10 Volumes Ethanol

Ladostigil tartrate (50 g) was dissolved in ethanol (500 ml, 10 vol.) by heating the solution to 45-58° C. The mixture was cooled to 0-5° C. gradually for 4.5 hours and was maintained at the same temperature for 2.5 hours. The product was collected by filtration and washed with 30 ml cold ethanol.

The wet material was determined to be polymorph C.

The wet material was dried at 60° C. and 70° C. under vacuum for 14 hours, giving polymorph A.

### Example 3

### Re-Crystallization of Ladostigil Tartrate in Tetrahydrofuran

Ladostigil tartrate (1.55 g) was dissolved in tetrahydrofuran (20 ml, 13 vol.) by heating the solution to 60° C. The mixture was cooled gradually to 26° C. for 4.5 hours. The product was collected by filtration.

The wet material was determined to be a mixture of polymorphs A and A1.

The material was dried under vacuum at ambient temperature for 14 hours, giving a mixture of polymorphs A and A1.

### Example 4

### Re-Crystallization of Ladostigil Tartrate in Acetonitrile

Ladostigil tartrate (0.38 g) is dissolved in acetonitrile (20 ml, 53 vol.) by heating the solution to 60° C. The mixture was cooled gradually to 26° C. for 5 hours. The product was collected by filtration.

The wet material was determined to be polymorph F.

The material was dried under vacuum at 80° C. for 14, giving a mixture of polymorphs A and A1.

### Example 5

### Re-Crystallization of Ladostigil Tartrate in Dioxane

Ladostigil tartrate (1.77 g) was dissolved in dioxane (20 ml, 11 vol.) by heating the solution to 54° C. The mixture was cooled gradually to room temperature over 5 hours. The product was collected by filtration.

The wet material was determined to be polymorph H with low crystallinity.

The wet material was dried under vacuum at 80° C. for 14 hours, giving a mixture of polymorphs A and A1.

### Example 6

### Re-Crystallization of Ladostigil Tartrate in 0.86 Volumes Water

Ladostigil tartrate (50 g) was dissolved in water (43 ml, 0.86 vol.) by heating the solution to 60° C. The mixture was seeded at 47° C. The temperature was maintained for 18 hours and the solution was cooled gradually to 20° C. for 4 hours. The product was collected by filtration.

The wet material was washed with isopropanol and was dried at room temperature under vacuum and was determined to be polymorph A.

The wet material was washed with isopropanol and dried at 80° C. under vacuum, giving polymorph A.

### Example 7

### Preparation of Ladostigil Tartrate in Ethanol (8.3 Volumes)

To a stirred solution of Ethyl-methyl-carbamic acid (R)-3-prop-2-ynylamino-indan-5-yl ester (74.6 g, 0.274 mole, 1 eq.) in absolute ethanol (335 ml, 265 gram) a warm solution of L-tartaric acid (21.6 g, 0.143 mole, 0.525 eq.) in absolute ethanol (158 ml, 125 gram) was added. The residue of tartaric acid in the vessel was washed with 126 ml (100 gram) absolute ethanol into the reaction mixture. The mixture was heated to 58-62° C. and filtered. The hot solution was divided in to four parts and returned to four reactors with different stirring devices (impeller and buffle), seeded at 37° C. and cooled to 0-5° C. gradually for 18 hours. The products from each reactor were collected by filtration and washed with cold ethanol.

The wet material was determined to be polymorph C. Some of the samples contained polymorph C with low crystallinity. The wet material was dried at 55° C. under vacuum for 17 hours, giving polymorph E. The wet polymorph with low crystallinity was transformed to polymorph E with low crystallinity upon drying under the same conditions. Both polymorph E and polymorph E with low crystallinity were transformed to form A upon standing.

Further drying of polymorph E under vacuum at 80° C. for 7 hours gave a mixture of polymorphs A1 and E, with primarily polymorph A1.

### Example 8

### Preparation of Ladostigil Tartrate in Ethanol (12 Volumes)

The same procedure as in Example 7 was repeated with a larger amount of ethanol.

The wet material was isolated and was determined to be polymorph C.

The wet material was dried at 80° C. under vacuum for 17 hours and polymorph E was formed. Polymorph E was transformed upon standing for a few hours to a mixture of polymorphs A and A1.

### Example 9

### Other Methods of Preparation of Form A

Re-crystallization of ladostigil tartrate in water-isopropanol mixture (1:1.4:13.6)

Ladostigil tartrate (38.9 g) was dissolved in water (53.5 ml, 1.4 vol.) by heating the solution to 45° C. Iso-propanol (530 ml, 13.6 vol.) was added to the solution and the heat was maintained for 2 hours. The mixture was cooled gradually to −3.5° C. (crystallization occurred below 0° C.) and the solution was maintained at the same temperature for 5 hours. The product was collected by filtration washed with 120 ml cold iso-propanol. One part of the wet material was dried at 25° C. under vacuum and was determined to be polymorph A.

Another part of the wet material was dried at 80° C. under vacuum and was determined to be polymorph A.

### Example 10

### Other Methods for the Preparation of Polymorph A Presented in a Tabulated Form

| | |
|---|---|
| 10-1 | Salt formation in EtOH:IPA (1:1) air drying at room temp |
| 10-2 | Salt formation in EtOH:IPA (3:1) air drying at room temp |

Other Polymorphic Forms

### Example 11

### Preparation of Ladostigil Tartrate in Ethanol

To a stirred solution of Ethyl-methyl-carbamic acid (R)-3-prop-2-ynylamino-indan-5-yl ester (200 g, 0.734 mole, 1 eq.) in absolute ethanol (1000 ml, 790 gram) a warm solution of L-tartaric acid (58 g, 0.386 mole, 0.525 eq.) in absolute ethanol (800 ml, 630 gram) was added. The residue of tartaric acid in the vessel was washed with 100 ml (80 gram) absolute ethanol which was added to the reaction mixture. The mixture was heated to 58-62° C. and filtered. The filtration system was washed with 100 ml (80 gram) absolute ethanol. The solution was returned to the reactor and was cooled to 0-5° C. gradually for 3-3.5 hours. The product was collected by filtration and washed with 150 ml cold ethanol and dried in a vacuum oven, first at 25° C. until constant weight was achieved, then at 50° C., and then at 80° C. to yield 210 g ladostigil tartrate (theoretical yield 87%).

The wet material was determined to be polymorph C.

The material dried at 25° C. under vacuum until constant weight was determined to be polymorph A1.

One part of the wet material was dried at 50° C. under vacuum for 12 hours, giving was polymorph B.

One part of the wet material was dried at 50° C. under vacuum for 12 hours, then at 80° C. under vacuum for 3 hours, giving a mixture of polymorphs consisting mainly of form A1 and a small amount of form E. Polymorph A1 is obtained upon further drying.

### Example 12

### Re-Crystallization of Ladostigil Tartrate in 5 Volumes Ethanol

Ladostigil tartrate (50 g) was dissolved in ethanol (250 ml, 5 vol.) by heating the solution to 55-62° C. The mixture was cooled to 0-5° C. gradually for 4.5 hours and was maintained at that temperature for 2.5 hours. The product was collected by filtration and was washed with 30 ml cold ethanol.

The wet material obtained was polymorph C.

One part of the wet material was dried at 60° C. under vacuum for 14 hours, giving a mixture of polymorphs A and A1.

One part of the wet material was dried at 60° C. under vacuum for 14 hours, then at 80° C. under vacuum for 3 hours, giving a mixture of polymorphs A1 and E, primarily polymorph A1.

In another experiment, the wet polymorph C obtained above was dried under vacuum at 50° C. for 20 hours and polymorph B was obtained. Further drying of the same material under vacuum at 80° C. for 3 hours resulted in a mixture of polymorphs A1 and E, primarily form A1.

### Example 13

### Re-Crystallization of Ladostigil Tartrate in 10 Volumes Ethanol

Ladostigil tartrate (50 g) was dissolved in ethanol (500 ml, 10 vol.) by heating the solution to 45-58° C. The mixture was cooled to 0-5° C. gradually for 4.5 hours and was maintained at that temperature for 2.5 hours. The product was collected by filtration and was washed with 30 ml cold ethanol.

The wet material was determined to be polymorph C.

One part of the wet material was dried at 60 and 70° C. under vacuum for 14 hours, giving polymorph A. Upon further drying for 3 hours under vacuum at 80° C., a mixture of polymorphs A1, E, and B was obtained, primarily polymorph A1.

Another part of the wet material was dried at 80° C. under vacuum for 3 hours, giving a mixture of polymorphs A1 and E.

When this mixture was dried at 90° C. under vacuum for 3 hours a mixture of polymorphs A1 and E was obtained, primarily polymorph A1.

### Example 14

### Preparation of Ladostigil Tartrate in 10 Volumes of Ethanol Containing 6% Toluene as a Co-Solvent

To a stirred solution of Ethyl-methyl-carbamic acid (R)-3-prop-2-ynylamino-indan-5-yl ester (59.6 g) in absolute ethanol (253 ml, 200 gram) a warm solution of L-tartaric acid (17.2 g) in absolute ethanol (190 ml, 150 gram) was added. The residue of tartaric acid in the vessel was washed with 63 ml (50 gram) absolute ethanol and was poured into the reaction mixture. The mixture was heated to 58-62° C. and filtered, and the filtration system was washed with 90 ml (71 gram) absolute ethanol. The resulted solution (“solution A”) was used in the co-solvent salt preparation experiments.

Toluene (6% relative to ethanol weight) was added and the solution was cooled in a controlled manner from 58° C. to 2° C. for 17 hours.

The wet material was collected by filtration and washed with cold ethanol. The wet material was determined to be polymorph C with low crystallinity.

One part of the wet material was dried at 55° C. under vacuum for 19 hours, giving polymorph B.

Another part of the wet material was dried at 55° C. under vacuum for 19 hours, then at 80° C. under vacuum for 7 hours, giving a mixture of polymorphs A1 and E, primarily form A1.

### Example 15

### Preparation of Ladostigil Tartrate in 10 Volumes of Ethanol Containing 2% Acetone as Co-Solvent

To the ethanolic solution A prepared as in example 14a, acetone (2% relative to ethanol weight) was added and the solution was cooled in a controlled manner from 60° C. to 2° C. for 20 hours.

The wet material was determined to be polymorph C.

One part of the wet material was dried at 55° C. under vacuum for 18 hours, giving polymorph B.

One part of the wet material was dried at 55° C. under vacuum for 18 hours, then at 80° C. under vacuum for 7 hours, giving a mixture of polymorphs A1 and E, primarily A1.

### Example 16

### Preparation of Ladostigil Tartrate in 10 Volumes of Ethanol Containing 2.25% Ethyl Acetate as a Co-Solvent

To the ethanolic solution A prepared as described in example 14a, ethyl acetate (2.25% relative to ethanol weight) was added and the solution was cooled in a controlled manner from 60° C. to 2° C. for 23 hours.

The wet material was determined to be polymorph C with low crystallinity.

One part of the wet material was dried at 55° C. under vacuum for 17 hours, giving polymorph B.

The wet material was dried at 55° C. under vacuum for 17 hours, then at 80° C. under vacuum for 7 hours, giving a mixture of polymorphs A1 and E, primarily A1.

### Example 17

### Preparation of Ladostigil Tartrate in 10 Volumes of Ethanol Containing 2% Dioxane as Co-Solvent

To the ethanolic solution A prepared as described in example 14a, dioxane 2% relative to ethanol weight was added and the solution was cooled in a controlled manner from 60° C. to 2° C. for 23 hours.

The wet material was determined to be polymorph C.

One part of the wet material was dried at 55° C. under vacuum for 17 hours, giving polymorph B.

One part of the wet material was dried at 55° C. under vacuum for 17 hours, then at 80° C. under vacuum for 7 hours, giving a mixture of polymorphs A1 and E, primarily A1.

### Example 18

### Re-Crystallization of Ladostigil Tartrate in 11 Volumes Acetone

Ladostigil tartrate (1.78 g) was dissolved in acetone (20 ml, 11 vol.) by heating the solution to 59° C. The mixture was seeded at 39° C. and cooled to 25° C. gradually. The product was collected by filtration.

The wet material was determined to be polymorph F with low crystallinity.

One part of the wet material was dried at 80° C. under vacuum for 17 hours, giving polymorph A1.

### Example 19

### Re-Slurry in 10 Volumes of Dioxane

Ethanol wet ladostigil tartrate polymorph C (20 g) was slurried in dioxane (200 ml) at 8-28° C. The product was collected by filtration.

The wet material was determined to be polymorph H.

One part of the wet material was dried at 80° C. under vacuum for 17 hours, giving polymorph A1.

### Example 20

### Re-Crystallization of Ladostigil Tartrate in 8 Volumes of Ethanol Containing 2.5% Water as Co-Solvent

Ladostigil tartrate (2.5 g) is dissolved in a mixture of ethanol (20 ml) and water (0.5 ml) by warming the solution to 60° C. The mixture is self seeded at 41° C. and cooled to 25° C. gradually. The product is collected by filtration.

The wet material was determined to be polymorph C.

The wet material was dried at ambient temperature under vacuum for 17 hours, then at 80° C. under vacuum for 7 hours, giving polymorph A1.

### Example 21

### Re-Crystallization of Ladostigil Tartrate in 8 Volumes of Ethanol Containing 0.2 Volumes of Water as a Co-Solvent

Ladostigil tartrate (18 g) was dissolved in a mixture of ethanol (144 ml) and water (3.6 ml) by warming the solution to 45° C. The mixture was seeded at 33° C. and cooled to 5° C. gradually over 6 hours. The product was collected by filtration and washed with 30 ml cold ethanol.

One part of the wet material was dried at ambient temperature under vacuum and was determined to be polymorph C.

One part of the wet material was dried under vacuum at 80° C., giving a mixture of polymorph A1 and E, primarily form A1.

### Example 22

### Re-Crystallization of Ladostigil Tartrate in 8 Volumes of Ethanol Containing 7.5% Methanol as a Co-Solvent

Ladostigil tartrate (2.5 g) is dissolved in a mixture of ethanol (20 ml) and acetic acid (0.5 ml) by warming the solution to 60° C. The mixture was cooled to 25° C. gradually. The product was collected by filtration.

The wet material was determined to be a mixture of polymorph A1 and E, primarily A1.

The wet material was dried at ambient temperature under vacuum for 17 hours, then at 80° C. under vacuum for 7 hours, giving polymorph A1.

### Example 23

### Slurry in Acetonitrile

Wet ladostigil tartrate recrystallized in ethanol (1:20) was slurried in 7.5 volumes of acetonitrile relative to the weight of the wet ladostigil tartrate for 1 hour. The solid was filtered at 52° C. The wet material was determined to be Form I.

The wet polymorph I was dried under vacuum at 80° C. The dried material was determined to be polymorph J.

### Example 24

### Lyophilization of Aqueous Ladostigil Tartrate Solution

Ladostigil tartrate (10 g) was dissolved in deionized water (300 ml) at ambient temperature. The solution was introduced into a stainless steel tray lyophilizer and cooled to less than −50° C. The lyophilizer condensers were cooled to −70° C. for 20 minutes and the temperature in the tray was raised to −40° C. Vacuum was maintained at 0.4-0.5 mmHg for 60 hours. Over the course of the 60 hours, the temperature in the tray was raised from −40° C. to −5° C. The lyophilization process was terminated by vacuum breaking. The lyophilized material was collected and was determined to be Form E.

One portion of the lyophilized material was dried in a vacuum oven at 55-60° C. until constant weight. The sample was determined to be Form E.

### Example 25

### Saturated Atmosphere Methylenechloride/Hexane

A saturated solution of ladostigil tartrate (conc. 0.12 g/ml) in methylene chloride was placed in a beaker that was contained in a chamber saturated with n-hexane vapors. The solution was kept in the chamber for 7 days. The crystals which formed on the wall of the beaker were collected and dried under vacuum without heating until constant weight was achieved. The crystals were determined to be form J1.

### Example 26

### Saturated Atmosphere DMF/Hexane

A saturated solution of ladostigil tartrate (conc. 0.35 g/ml) in dimethylformamide was placed in a beaker that is contained in a chamber saturated with n-hexane vapors. The solution was kept in the chamber for 7 days. The crystals which formed in the beaker were collected and dried under vacuum without heating until a constant weight was achieved. The crystals were determined to be form F.

### Example 27

### Saturated Atmosphere Acetone/Hexane

A saturated solution of ladostigil tartrate (conc. 1.6% w/w) in acetone was placed in a beaker that was contained in a chamber saturated with n-hexane vapors. The solution was kept in the chamber for 7 days. The crystals formed in the beaker were collected and dried under vacuum without heating until constant weight was achieved. The crystals were determined to be form F.

### Example 28

### Slurry in Acetone

A slurry of ladostigil tartrate form A1 (8 g) in acetone (30 ml, 3.75 vol.) was stirred at room temperature. After about 4 hours, a massive crystallization occurred that prevented effective stirring. Another portion of acetone (50 ml, 6.25 vol. total 10 volumes) was added. After a total of 24 hours stirring, the crystals were collected by filtration, washed with acetone, and dried under vacuum without heating until a constant weight (7 g) was achieved. The crystals were determined to be form F.

### Example 29

### Slurry in Acetone

A slurry of ladostigil tartrate polymorph A1 (8 g) in acetone (30 ml, 3.75 vol.) was stirred at room temperature. After about 4 hours, a massive crystallization occurred that prevented effective stirring. Another portion of acetone (50 ml, 6.25 vol. total 10 volumes) was added. After a total of 72 hours of stirring, the crystals were collected by filtration, washed with acetone, and dried under vacuum without heating to a constant weight (7.9 g). The crystals were determined to be form F

### Example 30

### Slurry in Acetonitrile

A slurry of ladostigil tartrate form A1 (8 g) in acetonitrile (30 ml, 3.75 vol.) was stirred at room temperature. After about 3 hours, a massive crystallization occurred that prevented effective stirring. Another portion of acetonitrile (50 ml, 6.25 vol. total 10 volumes) as added. After a total of 24 hours of stirring, the crystals were collected by filtration, washed with acetonitrile, and dried under vacuum without heating to a constant weight (7.5 g). The crystals were determined to be form F.

### Example 31

### Slurry in Acetonitrile

A slurry of ladostigil tartrate polymorph A1 (8 g) in acetonitrile (30 ml, 3.75 vol.) was stirred at room temperature. After about 3 hours, a massive crystallization occurred that prevented effective stirring. Another portion of acetonitrile (50 ml, 6.25 vol. total 10 volumes) was added. After a total of 72 hours of stirring, the crystals were collected by filtration, washed with acetonitrile, and dried under vacuum without heating to a constant weight (7.7 g). The crystals were determined to be form F.

### Example 32

### Slurry in Methylene Chloride

A slurry of ladostigil tartrate polymorph A1 (8 g) in methylene chloride (30 ml, 3.75 vol.) was stirred at room temperature. After about 2 hours, a massive crystallization occurred that prevented effective stirring. Another portion of methylene chloride (30 ml, 3.75 vol. total 7.5 volumes) was added. After a total of 24 hours stirring, the crystals were collected by filtration, washed with methylene chloride, and dried under vacuum without heating to constant weight (4 g). The crystals were determined to be form L.

### Example 33

### Slurry in Methylene Chloride

A slurry of ladostigil tartrate polymorph A1 (8 g) in methylene chloride (30 ml, 3.75 vol.) was stirred at room temperature. After about 2 hours, a massive crystallization occurred that prevented effective stirring. Another portion of methylene chloride (30 ml, 3.75 vol. total 7.5 volumes) was added. After a total of 72 hours stirring, the crystals were collected by filtration, washed with methylene chloride, and dried under vacuum without heating to a constant weight (4.4 g). The crystals were determined to be form L.

### Example 34

### Slurry in Dioxane

A slurry of ladostigil tartrate form A1 (8 g) in dioxane (30 ml, 3.75 vol.) was stirred at room temperature, after 21 hours another portion of dioxane (20 ml, 2.5 vol. total 6.25 volumes) was added. After a total of 24 hours stirring, the crystals were collected by filtration, washed with dioxane, and dried under vacuum without heating to a constant weight (7.8 g). The crystals were determined to be form L.

### Example 35

### Slurry in Dioxane

A slurry of ladostigil tartrate form A1 (8 g) in dioxane (30 ml, 3.75 vol.) was stirred at room temperature, after 21 hours another portion of dioxane (20 ml, 2.5 vol. total 6.25 volumes) was added. After a total of 72 hours of stirring, the crystals were collected by filtration, washed with dioxane, and dried under vacuum without heating to a constant weight (7.6 g). The crystals were determined to be form L.

### Example 36

### Slurry in THF

A slurry of ladostigil tartrate form A1 (8 g) in tetrahydrofuran (50 ml, 6.25 vol.) was stirred at room temperature for 72 hours. The crystals were collected by filtration, washed with tetrahydrofuran, and dried under vacuum without heating to a constant weight (4.5 g). The crystals were determined to be form L.

### Example 37

### Quench Cooling (Thermal Shock) of IPA Solution and Drying to Obtain Form B

A hot saturated solution of ladostigil tartrate (conc. 0.08 g/ml) in 2-propanol was prepared by dissolving at 60-70° C. is poured on a cold glass surface (−18° C., acetone/ice bath). The crystallization began after a few minutes and a massive crystallization occurred on the glass surface. The crystals formed were collected by filtration and were determined to be form B After drying under vacuum without heating until constant weight the crystals were determined to be form A1.

### Example 38

### Crystallization from Methylene Chloride to Obtain Form A

A saturated solution of ladostigil tartrate (conc. 0.15 g/ml) in methylene chloride was prepared by dissolving at room temperature. The solution was is stirred at room temperature for two hours. The crystals formed were collected by filtration, and washed with cold methylene chloride. The crystals were dried under vacuum without heating until a constant weight was achieved. The crystals were determined to be form A.

### Example 39

### Quench Cooling (Thermal Shock) of Methylene Chloride Solution to Obtain Essentially Amorphous Form

A saturated solution of ladostigil tartrate (conc. 0.14 g/ml) in methylene chloride was prepared by dissolving ladostigil tartrate at room temperature. The solution was poured on a cold glass surface (−18° C., acetone/ice bath). The solidification was not immediate, but after about half hour a solid formed on the glass surface. The solid formed was dried under vacuum without heating until constant weight. The solid was determined to be essentially amorphous.

### Example 40

### Purely Amorphous Form

10 g of ladostigil tartrate were dissolved in 90 ml methanol. The solution was subjected to spray drying. The solution was sprayed (290[ml/h]) into a chamber with hot nitrogen (38 m³/h, 70° C.) at co-current flow. The atomizing flow (500[l/h]) of nitrogen gave the droplets effect which lead to the high evaporation rate. The temperature of the outlet solids was fixed to 45° C. The sample was analyzed by XRD. Purely amorphous form was obtained.

### Example 41

Purely amorphous form of ladostigil tartrate was heated to 80° C. for 2 hours. The heated sample was analyzed by XRD. Form A1 was obtained.

### Example 42

### Water Uptake (%) and Crystal Form of Ladostigil Tartrate Form A and Form A1 Equilibrated at Different Relative Humidities

Crystal form was determined after equilibration of ladostigil tartrate form A for 10 days at various relative humidities at room temperature. The results are shown in table 8.

TABLE 8

Form A (equilibration time: 10 days)

| RH (%) | Crystal form | Water content |
|---|---|---|
| 0 | A | |
| 20 | A | 0.3 |
| 40 | A | 0.3 |
| 60 | A | 0.3 |
| 80 | A | 0.3 |
| 100 | A | 0.2 |

Form A was stable even at 100% relative humidity for 10 days.

In contrast, form A1 partially transforms to form A at 100% relative humidity, as seen below. Form A1 was exposed at various humidity conditions at room temperature for 13 days. The resulting crystal form and Karl Fischer analysis are summarized in table 9.

TABLE 9
Hygroscopiticity study of Form A1

| % RH | KF | Resulting form |
|---|---|---|
| 0 | 0.3 | |
| 20 | 0.3 | A1 |
| 40 | 0.3 | A1 |
| 60 | 0.3 | A1 |
| 80 | 0.3 | A1 |
| 100 | 0.5 | A1 + A |

### Example 43

### TGA and Crystal Form of Ladostigil Tartrate Form A1 and Various Humidity Conditions

Ladostigil tartrate form A1 was exposed to various humidity conditions at room temperature for 14 days. TGA was performed, and the crystal form was analyzed. The results are summarized in Table 10.

TABLE 10

| RH [%] | TGA | Resulting form |
|---|---|---|
| 60 | 0.2 | A1 |
| 80 | 0.1 | A1 |
| 100 | 0.1 | A1 + A |

Table 10 illustrates that pure form A1 was transformed to a mixture of Form A and A1 after exposure to 100% RH for 14 days, but the water content did not change significantly.

From these results it is evident that neither Form A nor Form A1 is hygroscopic.

### Example 44

### Stability of Mixture of Ladostigil Tartrate Mixture of Form A1 and Form E at High Humidity

A mixture of ladostigil tartrate form A1 and E, containing primarily form A1, was exposed at various humidity conditions at room temperature for 14 days. The results are summarized in Table 11.

TABLE 11
Hygroscopicity study of Form A1 + E

| % RH | Resulting Form |
|---|---|
| 60 | A1 > E |
| 80 | A1 > E |
| 100 | A1 > A |

Table 6 illustrates that when a mixture of form A1 and form E was exposed to 100% relative humidity, the mixture was transformed to a mixture of form A1 and A, primarily form A1.

### Example 45

### Stability of Form E at Different Humidity Conditions

Form E was placed for 7 days under 0-100% relative humidity at room temperature. Table 12 summarizes the results:

TABLE 12
Hygroscopicity of Form E

| % RH | Resulting Form |
|---|---|
| 0 | E |
| 20 | E |
| 40 | E |
| 60 | E |
| 80 | A1 > E |
| 100 | A1 > A |

Table 12 illustrates that form E is stable at 0-60% RH for one week. At 80% RH, Form E converts to form A1, and at 100% relative humidity Form E transforms to a mixture of form A1 and A, primarily form A1.

### Example 46

### Stability of Purely Amorphous Form at Different Humidity Conditions

Ladostigil tartrate purely amorphous form was placed for 8 days under 0-100% relative humidity at room temperature. Table 13 summarizes the results.

TABLE 13
Hygroscopicity study of Purely Amorphous form

| % RH | Resulting Form |
|---|---|
| 0 | Purely Amorphous |
| 20 | Purely Amorphous |
| 40 | Purely Amorphous |
| 60 | Purely Amorphous >>A1 |
| 80 | A1 |
| 100 | A |

Table 13 illustrates that purely amorphous form is stable at 0-40% RH for a minimum of 8 days. At 60% RH, traces of form A1 appear in the sample, and at 80% relative humidity, purely amorphous form transformed to form A1. At 100% relative humidity purely amorphous form transformed to form A.

### Example 47

### Stability of Form F at Different Humidity Conditions

Form F was placed for 14 days under 0-100% relative humidity at room temperature. Table 14 summarizes the results:

TABLE 14
Hygroscopicity study of Form F

| Relative | Water content by | Loss on Dry by | |
|---|---|---|---|
| humidity [%] | Karl Fisher [%] | TGA [%] | Crystal Form |
| 0 | 1.3 | Not measured | Form F |
| 20 | 2.2 | 3.0 | Form F |
| 40 | 2.6 | 2.9 | Form F |
| 60 | 0.3 | 0.3 | Form A1 + ˜10% E |
| 80 | 0.3 | 0.2 | Form A1 + ˜20% A |
| 100 | 0.3 | 0.3 | Form A + ˜20% A |

Table 14 illustrates that Form F is stable between about 0-40% RH for a minimum of 14 days about 60-100% RH, Form F transforms to Form E, A1 and A.

### Example 48

### Stability of Form H at Different Humidity Conditions

Ladostigil tartrate Form H was exposed for 14 days under 0-100% relative humidity at room temperature. Table 15 summarizes the results:

TABLE 15
Hygroscopicity study of Form H

| Relative | Water content by | Loss on | |
|---|---|---|---|
| humidity [%] | Karl Fisher [%] | Dry by TGA [%] | Crystal Form |
| 0 | 0.42 | 6.1 | Form H |
| 20 | 0.7 | 5.2 | Form F |
| 40 | 1.4 | 2.7 | Form L |
| 60 | 0.3 | 0.5 | Form A1 + ˜30% E |
| 80 | 0.2 | 0.2 | Form A1 + ˜20% A |
| 100 | 0.3 | 0.2 | Form A + ˜30% A1 |

Table 15 illustrates that Form H transforms to Form F on 20% RH, to Form L on 40% RH and to Form E, A1 and A on 60-100% RH. Form H may be used as an intermediate in A or A1.

### Example 49

### Stability of Form J1 at Different Humidity Conditions

Crystal ladostigil tartrate Form J1 was placed for 9 days under 0-100% relative humidity at room temperature. Table 16 summarizes the results:

TABLE 16
Hygroscopicity study of Form J1

| Relative | Water content by | Loss on Dry by | |
|---|---|---|---|
| humidity [%] | Karl Fisher [%] | TGA [%] | Crystal Form |
| 0 | 0.1 | 5.4 | Form J1 |
| 20 | | | Form K + ˜20% J1 |
| 40 | 0.2 | 0.8 | Form K |
| 60 | 0.2 | 0.5 | Form K |
| 80 | | | Form A1 + ˜20% A |
| 100 | 0.0 | 0.1 | Form A + ˜30% A1 |

Table 16 illustrates that Form J1 transforms partly to Form K at 20% RH, to Form K at 40-60% RH and to Form A1 and A at 80-100% RH. Form K is stable at 20-60% RH, and may be used for administration in pharmaceutical compositions. Further, Form J1 may be used as an intermediate in preparation of Form K, A or A1.

### Example 50

### Effect of Heating

Various polymorph forms and mixtures thereof were heated. The heating conditions and resulting polymorph forms are described in table 17 below:

TABLE 17

| Starting | | Time | Resulting |
|---|---|---|---|
| form | Experimental conditions | (hours) | form |
| A1 + A | RT under vacuum | | A1 + A |
| | 55° C. under vacuum | 17 | A1 + A |
| A | 130 C. | 3 | A1 |
| A1 | RT under vacuum | | A1 |
| | 50° C. under vacuum | 12 | A1 |
| | 55° C. | 17 | A1 |
| | RT for 17 hours, then 80° C. | 7 | A1 |
| | 80° C. | | A1 |
| | 130 C. | 3 | A1 |
| B | 80° C. | 6 | A1 + E |
| | 80° C. | 9 | A1 + E |
| | 80° C. | 17 | A1 + E |
| C | 50° C. under vacuum | 12 | B |
| | 55° C. | 17 | B |
| | 55° C. | 19 | B |
| | 55° C. under vacuum | 17 | E |
| | 50° C. under vacuum | 1 | A1 + C |
| | 50° C. under vacuum for 20 hours | 4 | A1 + E |
| | then, 80° C. | | |
| | 50° C. under vacuum for 12 hours | 3 | A1 + E |
| | then 80° C. | | |
| F | 80° C. under vacuum | | A1 + A |
| | 80° C. under vacuum | | A1 |
| H | 80° C. under vacuum | | A1 + A |
| | 80° C. under vacuum | | A1 |
| J1 | 60° C. in conventional oven | 2 | A1 + J1 |
| | 100° C. in conventional oven | 2 | A1 |
| K | 60° C. in conventional oven | 2 | K |
| | 100° C. in conventional oven | 2 | K |
| L | 60° C. in conventional oven | 2 | L |
| | 100° C. in conventional oven | 2 | A1 |
| Purely | 80° C. in conventional oven | 2 | A1 + A |
| amorphous | | | |

Form A+A1 (30 and 80% Form A content) dried at 55° C. retained the same polymorphic content of A+A1.

At 80° C. Form B transformed to a mixture of Form A1 and form E, primarily form A1. There were no significant differences in form E concentration between the samples dried for 6 or 9 or 17 hours at 80° C.

Form C transformed to different mixtures of A1, B, C, and E as a function of drying temperature and drying time. After 1 hour of drying at 50° C. Form C was still detected. After drying for 12 hours at 50° C. under vacuum, or for 17-19 hours at 55° C., Form B was formed. After drying for 17 hours at 55° C. under vacuum Form E was obtained.

Forms F and H transformed to mixtures of Form A1 and A at 80° C. under vacuum.

Forms J1 transformed partly to Form A1 at 60° C. Form L at 60° C. polymorphically stable minimum for 3 hours of storage time.

Forms J1 and L transformed to Form A1 at 100° C.

Purely amorphous form transformed to mixtures of Form A1 and A at 80° C.

Forms A transformed to Form A1 at 130° C.

### Example 51

### Effect of Micronization, Pressing and Grinding

The effects of micronization, pressing (1 minute at 100 metric tons) and grinding were determined by XRD and the following results were obtained:

Form A upon grinding transformed to a mixture of Forms A and A1, and after pressing the form A transformed mainly to Form A1. Crystallinity degradation was also observed upon pressing. Form A and Form A1 lose some extent of crystallinity after pressing. The XRD peaks become broader and the intensities became smaller after grinding and pressing the sample.

### Example 52

### Effect of Freezing

The effects of freezing was determined by XRD and the following results were obtained:

Form A1 was kept in a freezer at about −25° C. for 3 months. Form A1 transformed to Form A in freezer.

What is claimed is:

1. Crystalline ladostigil tartrate characterized by an x-ray diffraction pattern having peaks at 19.5, 22.9, and 23.1±0.2 degrees two theta.

2. Crystalline ladostigil tartrate of claim 1 further characterized by an x-ray diffraction pattern having a peak at 18.4±0.2 degrees two theta.

3. Crystalline ladostigil tartrate of claim 2 further characterized by an x-ray diffraction pattern having peaks at 8.6, 13.8, and 18.0±0.2 degrees theta.

4. Crystalline ladostigil tartrate of claim 1 having an XRD pattern as substantially depicted in FIG. 9.

5. A process for preparing the crystalline form of claim 1 comprising crystallizing ladostigil tartrate from a solution of ladostigil tartrate in water, tetrahydrofuran, isopropanol, methylene chloride, or mixtures thereof, and recovering the crystalline form.

6. The process of claim 5, wherein crystallizing ladostigil tartrate comprises the steps of:
a) providing a solution of ladostigil tartrate in water, tetrahydrofuran, isopropanol, methylene chloride, or mixtures thereof;
b) cooling the solution to crystallize ladostigil tartrate; and
c) recovering the ladostigil tartrate.

7. The process of claim 6, wherein the solution of ladostigil tartrate is provided by heating ladostigil tartrate in a solvent selected from the group consisting of water, tetrahydrofuran, isopropanol, methylene chloride, and mixtures thereof to obtain a solution.

8. The process of claim 6, wherein cooling is from a temperature of at least about 40° C. to a temperature of below about 20° C.

9. A process for preparing the crystalline ladostogil tartrate of claim 1 comprising crystallizing ladostigil tartrate from a solution of ladostigil tartrate in ethanol, acetonitrile, dioxane, or mixtures thereof, to obtain a wet crystal, heating the wet crystal to obtain the crystalline form, and recovering the crystalline form.

10. The process of claim 9, wherein heating is carried out at a temperature of about 40° C. to about 100° C.

11. A crystalline form of ladostigil tartrate, wherein the crystalline form does not transform to another crystalline form after exposure to air having relative humidity of 100% for 10 days.

12. Crystalline ladostigil tartrate characterized by an x-ray diffraction pattern having peaks at 4.3, 5.6, 11.2, 13.0, 16.8, and 19.9±0.2 degrees two theta.

13. Crystalline ladostigil tartrate of claim 12, having an XRD pattern as substantially depicted in FIG. 17.

14. A process for preparing the crystalline ladostigil tartrate of claim 12 comprising crystallizing ladostigil tartrate from a solution of ladostigil tartrate in a C₁ to C₄ alcohol by rapid precipitation and recovering the crystalline form.

15. The process of claim 14, wherein rapid precipitation comprises the steps of:
a) heating a solution of ladostigil tartrate in a C₁ to C₄ alcohol; and
b) rapid cooling of the solution to obtain crystalline ladostigil tartrate.

16. The process of claim 15, wherein the alcohol is 2-propanol.

17. The process of claim 15, wherein heating is to a temperature of at least about 40° C.

18. The process of claim 15, wherein step b) comprises combining or contacting the solution with a cold solid or liquid having a temperature of at least 30° C. below that of the solution to obtain crystalline ladostigil tartrate.

19. A process for preparing the crystalline form of claim 12 comprising crystallizing ladostigil tartrate from a solution of ladostigil tartrate in 2-propanol by combining the solution with a solid surface having a temperature below that of the solution to precipitate the crystalline form within about 1 hour of combining, and recovering the crystalline form.

20. A process for preparing the crystalline form of claim 12 comprising crystallizing ladostigil tartrate from a solution of ladostigil tartrate in a C₁ to C₄ alcohol by combining the solution with an anti-solvent to precipitate the crystalline form within about 1 hour of combining, and recovering the crystalline form.

21. The process of claim 20, wherein the anti-solvent is a C₅ to C₇ cyclic or acyclic saturated hydrocarbon.

22. Crystalline ladostigil tartrate characterized by an x-ray diffraction pattern having peaks at 5.8, 10.8, 13.3, 17.4, 23.6±0.2 degrees two theta.

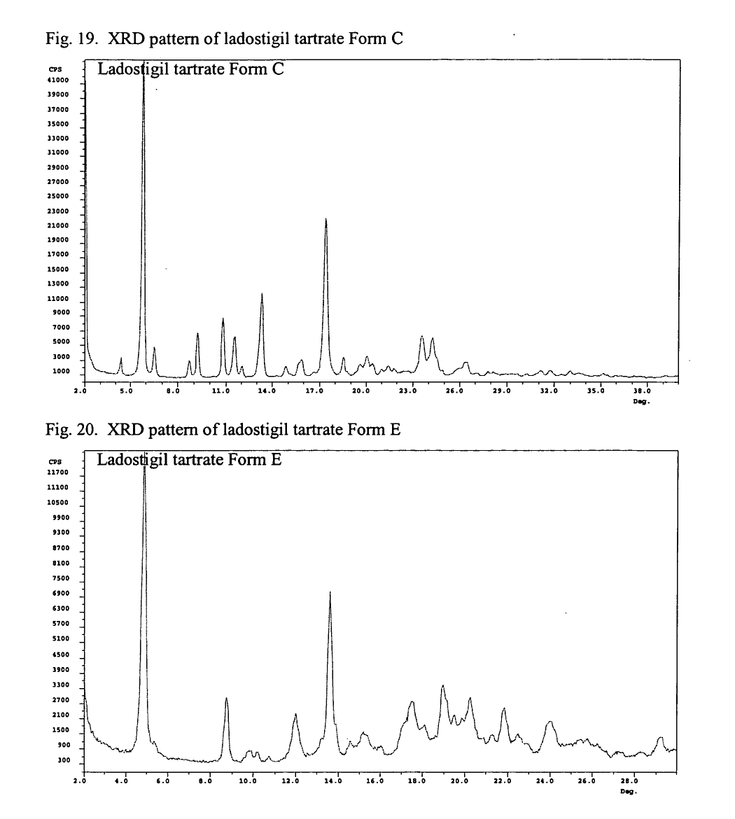

23. Crystalline ladostigil tartrate of claim 22 having an XRD pattern as substantially depicted in FIG. 19.

24. A process for preparing crystalline form of claim 22 comprising crystallizing ladostigil tartrate from a solution of ethanol and recovering the crystalline form.

25. The process of claim 24, wherein the ethanol contains another solvent up to about 20% by volume.

26. The process of claim 25, wherein crystallizing comprises the steps of:
a) providing a solution of ladostigil tartrate in ethanol;
b) heating the solution; and
c) cooling the solution to obtain the crystalline form.

27. The process of claim 26, wherein step a) comprises dissolving ladostigil tartrate in ethanol, or dissolving ladostigil base and tartaric acid in ethanol.

28. The process of claim 26, wherein heating is to a temperature of about 40° C. to about 60° C.

29. The process of claim 26, wherein cooling is to a temperature of about 0° C. to about 25° C.

30. Crystalline ladostigil tartrate characterized by an x-ray diffraction pattern having peaks at 4.9, 8.7, 12.0, 13.6, and 18.9±0.2 degrees two theta.

31. Crystalline ladostigil tartrate of claim 30 having an XRD pattern as substantially depicted in FIG. 20.

32. A process for preparing crystalline form of claim 30 comprising lyophilizing the crystalline form from an aqueous solution of ladostigil tartrate.

33. The process of claim 32, wherein the aqueous solution consists of water as a solvent.

34. Crystalline ladostigil tartrate characterized by an x-ray diffraction pattern having peaks at 3.3, 6.4, 13.0, 13.3, and 19.6±0.2 degrees two theta.

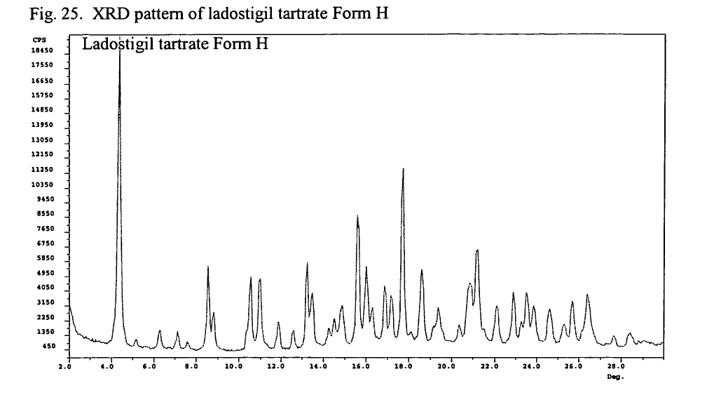

35. Crystalline ladostigil tartrate of claim 34 having an XRD pattern as substantially depicted in FIG. 24.

36. A process for preparing the crystalline form of claim 34 comprising slurrying crystalline ladostigil tartrate characterized by an XRD pattern having peaks at 8.7, 13.9, and 17.4±0.2 degrees 2 theta, in DMF, acetone, hexane, acetonitrile, or mixtures thereof to obtain the crystalline form, and recovering the crystalline form.

37. A process for preparing the crystalline form of claim 34 comprising crystallizing the crystalline form of ladostigil tartrate from a solution of ladostigil tartrate in DMF, acetone, acetonitrile, or mixtures thereof, by combining the solution with a C₅ to C₇ saturated hydrocarbon and recovering the crystalline form.

38. The process of claim 37, wherein the hydrocarbon is hexane.

39. The process of claim 37, wherein the hydrocarbon is heptane.

40. Crystalline ladostigil tartrate characterized by an x-ray diffraction pattern with peaks at 4.4, 8.5, 10.5, 15.6 and 17.7-±0.2 degrees two theta.

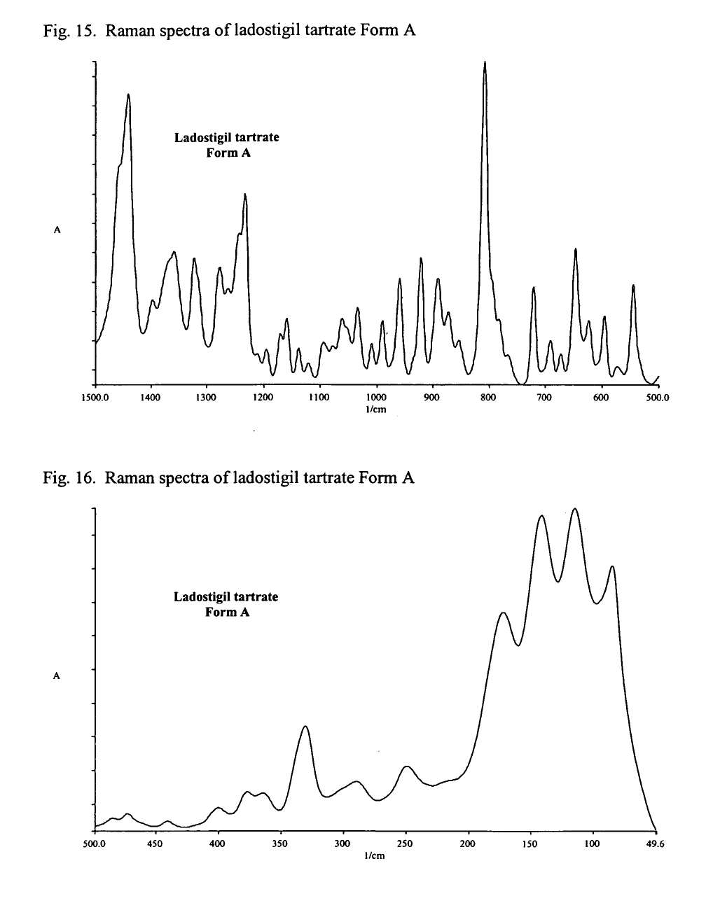

41. Crystalline ladostigil tartrate of claim 40 having an XRD pattern as substantially depicted in FIG. 15.

42. A process for preparing crystalline form of claim 40 comprising crystallizing the crystalline form of ladostigil tartrate from a solution of ladostigil tartrate in dioxane and recovering the crystalline form.

43. The process of claim 42, wherein crystallizing comprises heating the solution of ladostigil tartrate in dioxane and cooling the solution to obtain the crystalline form.

44. The process of claim 43, wherein heating is to a temperature of at least about 40° C. and cooling is to a temperature below about 20° C.

45. Crystalline ladostigil tartrate characterized by an x-ray diffraction pattern with peaks at 6.5, 12.0, 13.0, 13.3 and 18.6±0.2 degrees two theta.

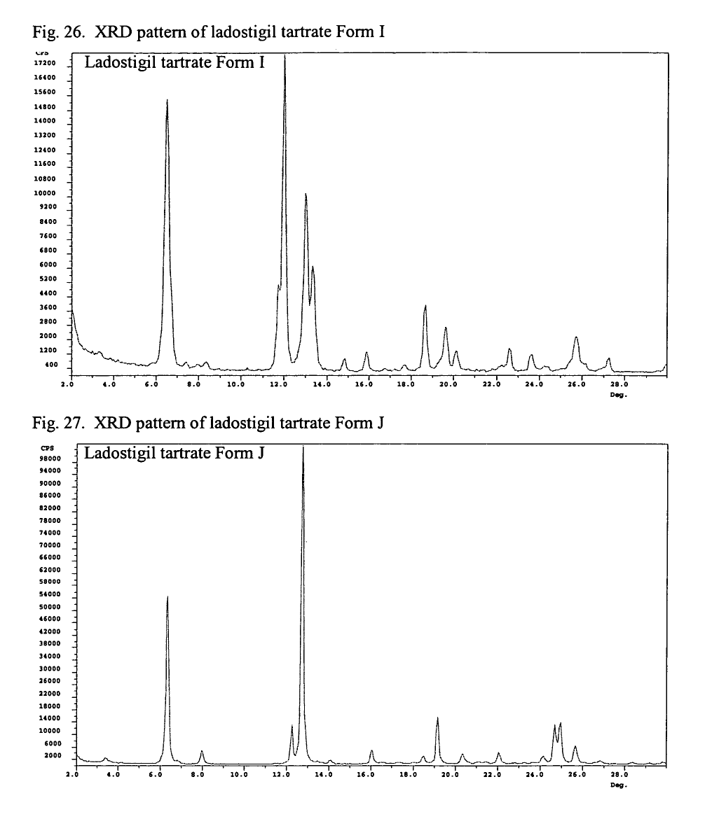

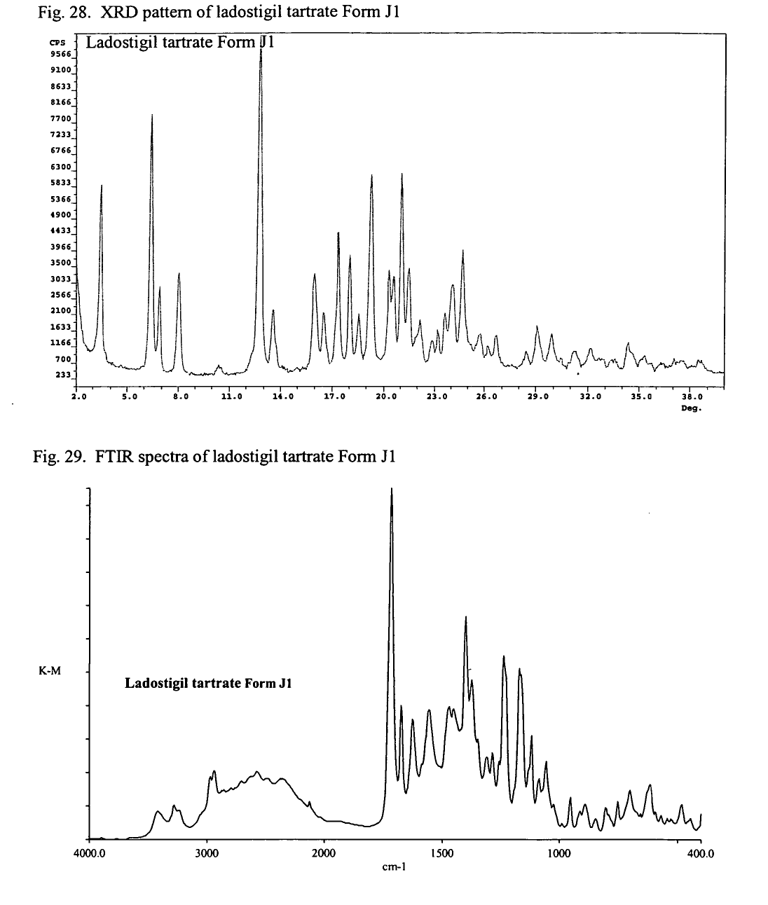

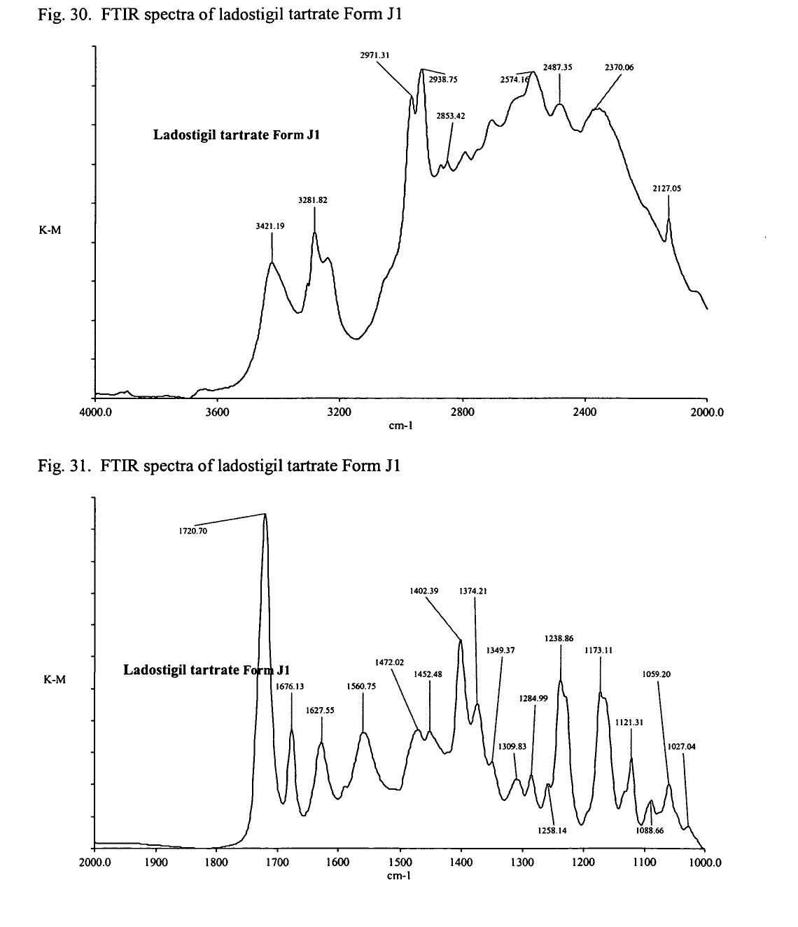

46. Crystalline ladostigil tartrate of claim 45 having an XRD pattern as substantially depicted in FIG. 26.

47. A process for preparing crystalline form of claim 45 comprising maintaining a heterogeneous mixture of ladostigil tartrate in acetonitrile to obtain the crystalline form and recovering the crystalline form.

48. The process of claim 47, wherein the crystalline form in the mixture is obtained by recrystallization from ethanol.

49. Crystalline ladostigil tartrate characterized by an x-ray diffraction pattern having peaks at 6.3, 12.2, 12.7 and 24.7±0.2 degrees two theta.

50. Crystalline ladostigil tartrate of claim 49 having an XRD pattern as substantially depicted in FIG. 27.

51. Crystalline ladostigil tartrate characterized by an x-ray diffraction pattern having peaks at 3.5, 6.5, 12.8, 19.3, and 21.1 degrees two theta.

52. Crystalline ladostigil tartrate of claim 51 having an XRD pattern as substantially depicted in FIG. 28.

53. A process for preparing the crystalline form of claim 51 comprising crystallizing ladostigil tartrate from a solution of ladostigil tartrate in methylenechloride by combining the solution with a C₅ to C₇ saturated hydrocarbon and recovering the crystalline form.

54. The process of claim 53, wherein the hydrocarbon is hexane or heptane.

55. Crystalline ladostigil tartrate characterized by an x-ray diffraction pattern having peaks at 6.4, 12.1, 12.8, and 14.6±0.2 degrees two theta.

56. Crystalline ladostigil tartrate of claim 55 having an XRD pattern as substantially depicted in FIG. 21.

57. A process for preparing the crystalline form of claim 55 comprising placing Form J1 under 10-70% RH for a sufficient time to obtain the crystalline form.

58. Crystalline ladostigil tartrate characterized by an x-ray diffraction pattern having peaks at 4.5, 8.9, 11.4, 14.6, and 18.4±0.2 degrees two theta.

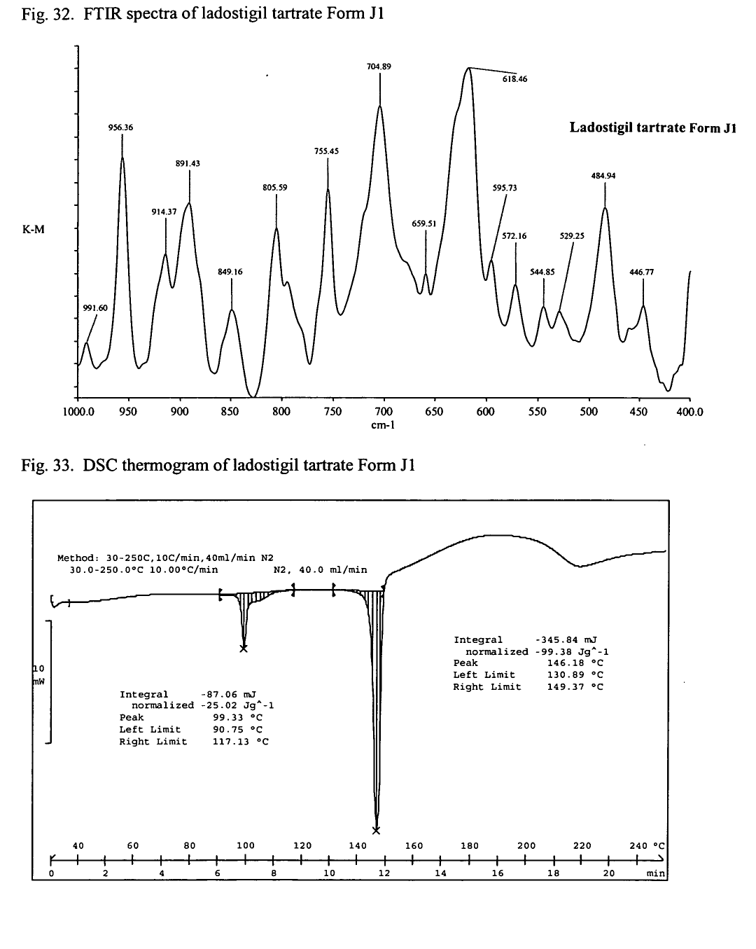

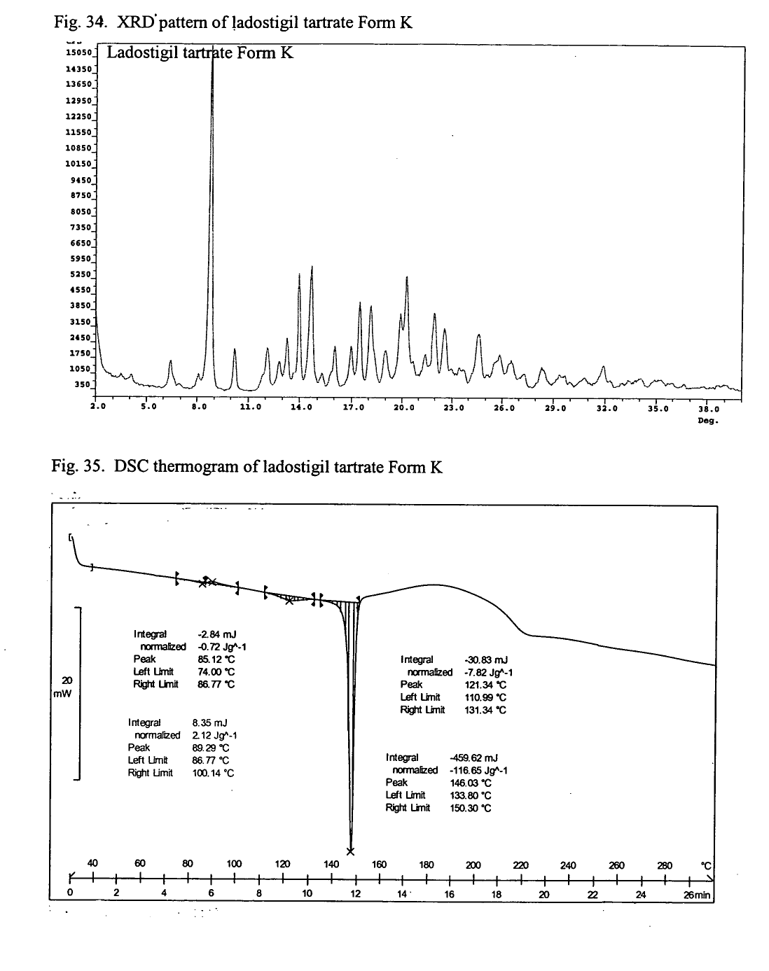

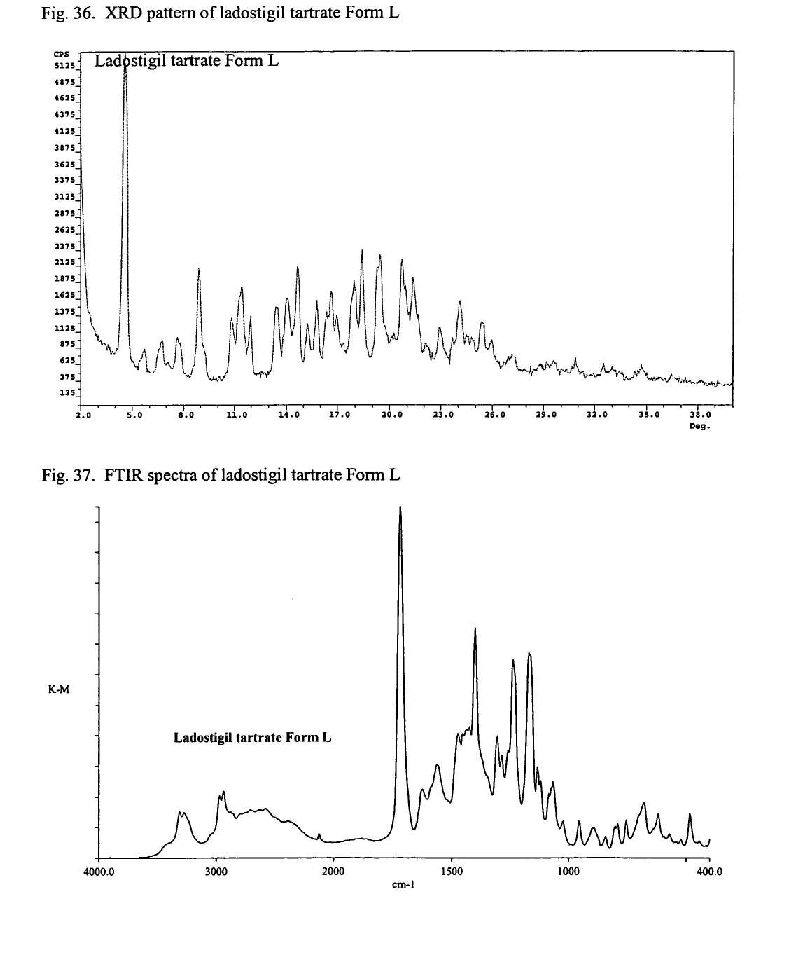

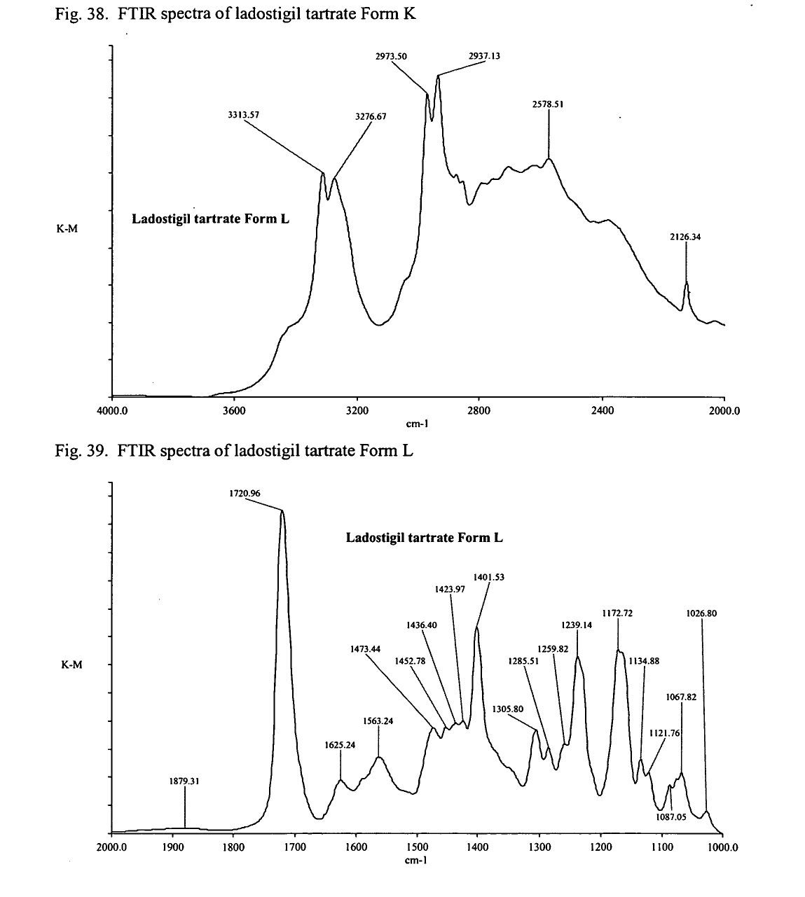

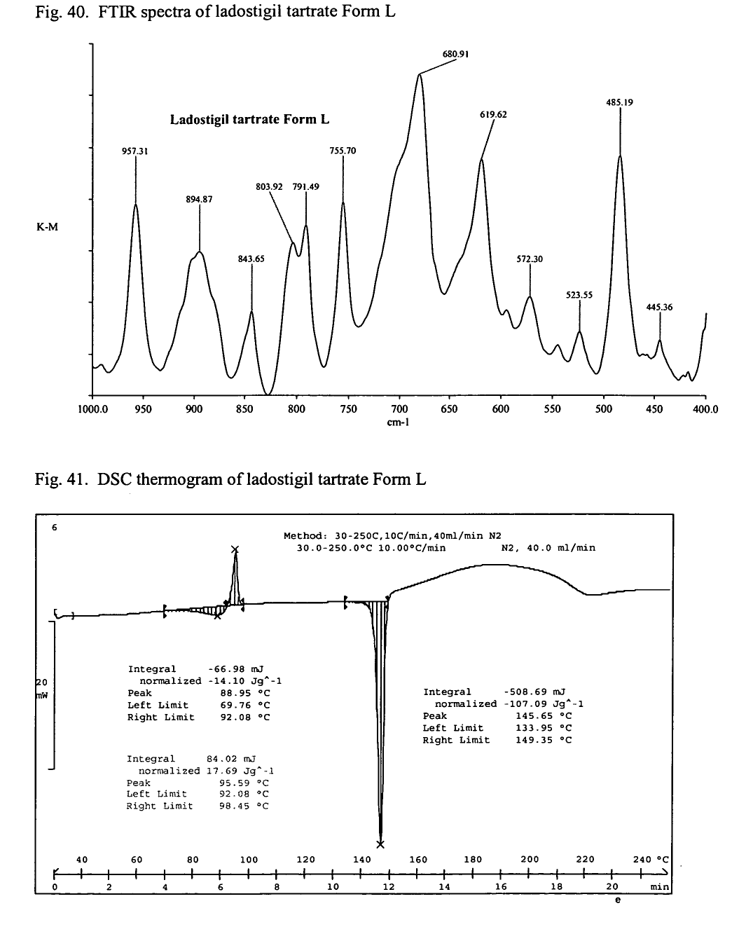

59. Crystalline ladostigil tartrate of claim 58 having an XRD pattern as substantially depicted in FIG. 32.

60. A process for preparing crystalline form of claim 58 comprising maintaining a heterogeneous mixture of crystalline ladostigil tartrate in a solvent selected from the group consisting of CH₂Cl₂, dioxane, THF, and mixtures thereof to obtain the crystalline form and recovering the crystalline form.

61. Solid ladostigil tartrate in amorphous form.

62. Amorphous ladostigil tartrate of claim 61 having an XRD pattern as substantially depicted in FIG. 51.

63. The amorphous form of claim 62, wherein the amorphous form is present in a composition having a crystalline content of less than about 10% by XRD.

64. The amorphous form of claim 63, wherein the amorphous form is present in a composition having a crystalline content of less than about 5% by XRD.

65. The amorphous form of claim 65, wherein the amorphous form is present in a composition having a crystalline content of less than about 1% by XRD.

66. A process for preparing amorphous form of claim 61 comprising spray drying a solution of ladostigil tartrate in a C₁ to C₄ alcohol or in water.

67. The process of claim 66, wherein the alcohol is methanol.

68. A process for preparing essentially amorphous ladostigil tartrate comprising precipitating ladostigil tartrate from a solution of ladostigil tartrate in dimethylene chloride by combining or contacting the solution with a cold solid or liquid having a temperature of at least 20° C. below that of the solution and recovering the amorphous form.

69. A process for preparing crystalline ladostigil tartrate characterized by an x-ray diffraction pattern having peaks at 19.5, 22.9, and 23.1±0.2 degrees two theta, comprising heating crystalline ladostigil tartrate characterized by an x-ray diffraction pattern having peaks at 3.3, 6.4, 13.0, 13.3, and 19.6±0.2 degrees two theta, or crystalline ladostigil tartrate characterized by an x-ray diffraction pattern with peaks at 4.4, 8.5, 10.5, 15.6 and 17.7±0.2 degrees two theta.

70. A process for preparing crystalline ladostigil tartrate characterized by an x-ray diffraction pattern having peaks at 19.5, 22.9, and 23.1±0.2 degrees two theta, comprising cooling crystalline ladostigil tartrate characterized by an XRD pattern having peaks at 8.7, 13.9, and 17.4±0.2 degrees 2 theta.

71. A process for preparing crystalline ladostigil tartrate characterized by an x-ray diffraction pattern having peaks at 4.9, 8.7, 12.0, 13.6, and 18.9±0.2 degrees two theta, comprising heating crystalline ladostigil tartrate characterized by an x-ray diffraction pattern having peaks at 4.3, 5.6, 11.2, 13.0, 16.8, and 19.9±0.2 degrees two theta, or crystalline ladostigil tartrate characterized by an x-ray diffraction pattern having peaks at 5.8, 10.8, 13.3, 17.4, 23.6±0.2 degrees two theta.

72. A process for preparing crystalline ladostigil tartrate characterized by an x-ray diffraction pattern having peaks at 4.3, 5.6, 11.2, 13.0, 16.8, and 19.9±0.2 degrees two theta, comprising heating crystalline ladostigil tartrate characterized by an x-ray diffraction pattern having peaks at 5.8, 10.8, 13.3, 17.4, 23.6±0.2 degrees two theta.

73. A process for preparing crystalline ladostigil tartrate characterized by an x-ray diffraction pattern having peaks at 19.5, 22.9, and 23.1±0.2 degrees two theta, comprising exposing crystalline ladostigil tartrate characterized by an XRD pattern having peaks at 8.7, 13.9, and 17.4±0.2 degrees 2 theta to high relative humidity.

74. The process of claim 73, wherein the exposure is carried out to an atmosphere containing a relative humidity of about 90% to about 100%.

75. The process of claim 74, wherein the humidity is about 100%.

76. A process for preparing crystalline ladostigil tartrate characterized by an x-ray diffraction pattern having peaks at 19.5, 22.9, and 23.1±0.2 degrees two theta, or crystalline ladostigil tartrate characterized by an XRD pattern having peaks at 8.7, 13.9, and 17.4±0.2 degrees 2 theta, comprising exposing amorphous ladostigil tartrate to high relative humidity.

77. The process of claim 76, wherein exposure is carried out to an atmosphere containing at least about 60% relative humidity.

78. The process of claim 77, wherein the relative humidity is about 60% to about 90%, resulting in crystalline ladostigil tartrate characterized by an XRD pattern having peaks at 8.7, 13.9, and 17.4±0.2 degrees 2 theta.

79. The process of claim 77, wherein the relative humidity is about 90% to about 100%, resulting in crystalline ladostigil tartrate characterized by an x-ray diffraction pattern having peaks at 19.5, 22.9, and 23.1±0.2 degrees two theta.

80. Crystalline ladostigil tartrate solvate.

81. The crystalline ladostigil tartrate solvate of claim 80, wherein the solvate is a dioxane solvate.

82. The crystalline ladostigil tartrate solvate of claim 80, wherein the solvate is an acetonitrile solvate.

83. The crystalline ladostigil tartrate solvate of claim 80, wherein the solvate is a methylenechloride solvate.

84. The crystalline ladostigil tartrate solvate of claim 80, wherein the solvate is an ethanol solvate.

85. The crystalline ladostigil tartrate solvate of claim 80, wherein the solvate is a tetrahydrofuran solvate.

86. Crystalline ladostigil tartrate hydrate.

87. The crystalline ladostigil tartrate hydrate of claim 86, wherein the hydrate is a hemihydrate.

88. The crystalline ladostigil tartrate hydrate of claim 86, wherein the hydrate is a monohydrate.

89. A pharmaceutical composition comprising a therapeutically effective amount of ladostigil tartrate selected from the group consisting of A, B, C, E, F, H, I, J, J1, K, L and amorphous form and a pharmaceutically acceptable carrier.

90. A process of preparing a pharmaceutical composition comprising the step of combining ladostigil tartrate selected from the group consisting of A, B, C, E, F, H, I, J, J1, K, L and amorphous form, or a solution prepared with one or more of these forms, with a pharmaceutically acceptable carrier.

91. A method of treating Alzheimer's disease comprising administering to a human subject in need thereof the pharmaceutical composition of claim 89.

92. A method of treating a mammal in need of inhibition of the acetylcholine esterase enzyme comprising administering the pharmaceutical composition of claim 89 to the mammal.

93. A method of treating a mammal in need of inhibition of the monoamine oxidase type B enzyme comprising administering the pharmaceutical composition of claim 89 to the mammal.
